SEQUENCE LISTING (12 sequences)

SEQ ID NO: 1 - protein, 107 aa, Human
MARATLSAAPSNPRLLRVALLLLLLVAASRRAAGAPLATELRCQCLQTLQGIHLKNIQSVKVKSPGPHCAQTEVIATLKNGQKACLNPASPMVKKIIEKMLKNGKSN

SEQ ID NO: 2 - protein, 100 aa, Mouse
MAPPTCRLLSAALVLLLLLATNHQATGAVVASELRCQCLKTLPRVDFKNIQSLSVTPPGPHCAQTEVIATLKGGQKVCLDPEAPLVQKIIQKILNKGKAN

SEQ ID NO: 3 - DNA, 2259 bp, Human
cgggatcgatctggagctccgggaatttccctggcccgggactccgggctttccagccccaaccatgcataaaaggggttcgccgttctcggagagccacagagcccgggccacaggcagctccttgccagctctcctcctcgcacagccgctcgaaccgcctgctgagccccatggcccgcgccacgctctccgccgcccccagcaatccccggctcctgcgggtggcgctgctgctcctgctcctggtggccgccagccggcgcgcagcaggtgggtcccggcgccctggggtccccgggccggacgcggctggggtgggcgccccgcgccgacagccccgctcaatcagcgagtctcttcttccctaggagcgcccctggccactgaactgcgctgccagtgcttgcagaccctgcagggaattcacctcaagaacatccaaagtgtgaaggtgaagtcccccggaccccactgcgcccaaaccgaagtcatgtaagtctcgcccatcgctgccgctgccactgctggggtccccgactctcccgctgccccaaaccctgtcctcagcccgacctcctgtctcacgagattcccttctctctgcagagccacactcaagaatgggcagaaagcttgtctcaaccccgcatcgcccatggttaagaaaatcatcgaaaagatgctgaaaaagtaagttataatttccatgtacacaggcgactggagctgttggtcagaaatactggcgtctgccccctaaaaagtaaatcaggaaaacccagggttagctgcaggactgaaaaaattattattttcacaaagttgccattaaggttattaatctgttctggtgccagaggatattcccagtgcccagggtcaccacccaggttcttccctcctgccagtgaatgtaggtaaaactgctttcatgtaaggtctcattggagtttgccagttgtgctgaagtgttttccacctgttaaaaaacacaagcttcatttcccctagttcctactgactttcagacctgaaaagcagaccccctggttttttgtaaaagtttagggttgaagtgcagagatttcacaataatattgcactgggtatataactggttgccaccacctattagccatgtcactcagagacaatattatttagtttatctgagactcagcaaaacagggaaaataatggtagctacatcatgaggttgctttgaggctcaagtgaggcccagtatgacaaagagcaataggctctggctgttttagccagtaaaacagtgtagactgccactcctccaccccacgtcacccccattcctaaaagagcatcccaagcctagaggtccttgccacacagcacagctgtcacaggcagtacccacttgggtgccaggctggggaaactgcattcagaaaactctagagcctggggaaacaggacaggagaagactgttgtgcagtcagctttcctgagcacctactcagggcacccattttctcattacagtggcaaatccaactgaccagaaggaaggaggaagcttattggtggctgttcctgaaggaggccctgcccttacaggaacagaagaggaaagagagacacagctgcagaggccacctggattgcgcctaatgtgtttgagcatcacttaggagaagtcttctatttatttatttatttatttatttgtttgttttagaagattctatgttaatattttatgtgtaaaataaggttatgattgaatctacttgcacactctcccattatatttattgtttattttaggtcaaacccaagttagttcaatcctgattcatatttaatttgaagatagaaggtttgcagatattctctagtcatttgttaatatttcttcgtgatgacatatcacatgtcagccactgtgatagaggctgaggaatccaagaaaatggccagtgagatcaatgtgacggcagggaaatgtatgtgtgtctattttgtaactgtaaagatgaatgtcagttgttatttattgaaatgatttcacagtgtgtggtcaacatttctcatgttgaagctttaagaactaaaatgttctaaatatcccttggacattttatgtctttcttgtaaggcatactgccttgtttaatgttaattatgcagtgtttccctctgtgttagagcagagaggtttcgatatttattgatgttttcacaaagaacaggaaaataaaatatttaaaaatataaaa

SEQ ID NO: 4 - DNA, 2066 bp, Mouse
agactgcgcctcagtgcctccaacaagcttcctcgggcactccagactccagccacacttcagcctagcgccatggcccctcccacctgccggctcctcagtgctgcactggtcctgctgctgctgctggccaccaaccaccaggctacaggtgggattcggattcgaggctgatgtttctggggaagcctcaggtgggcgcagccatccccagcccttctgacccagtgtcttctcctacaggggctgttgtggccagtgaactgcgctgtcaatgcctgaagaccctgccaagggttgacttcaagaacatccagagcttgagtgtgacgcccccaggaccccactgcgcccagacagaagtcatgtgagtgtctccctggatagcctctgccatggccagagttgcccagaccctcctgccgccccatgctcaccctaaagaaacctcgtgccttataatgatccccacatctctctgcagagccactctcaagggcggtcaaaaagtttgccttgaccctgaagcccccctggttcagaaaatcatccaaaagatactgaacaagtgagtcgcgatttgtgcttacacctgactggcacccgatttctgaaaattatagcagggaaaatcaaggttaacttcagttcaggaacctgaattattattttcattgttaactttgtcctgtgagcatcagtttgaattgatagaagggtacgactagctcctttcatctacacacgtaaaagtccttccttaatggatggtcgctgtgtgtccctaagaattaagtagatagaaccaactagcgctggtgtaaagcctgttatctattaagattcaatactttacccctgccctttccatgtctgtgggctgtaaagtctaactcttggttttggtcaaggtgagcctctaaggcttcccgatgaagaagagctggggtttccagtgagaggcttgaaattcaggtgtaggctctcgctcttatcctgactgagttcagtgaaaaagcaataggtattgttcttgtttacaacgtacgcgaagtcatgccttgacatttttactcttaacatcatttggaaaggttcaagctggtacaattgtaacgccagctctactattctagtttcaaagcgcccagagtgtttattgctggaaagtccagcaataggtagcaggtcccttgtggaggctagagatggtttcgtttggagagtaaaccaaggtccaggatgaagtcaaaagaagggggctctgcaacccgcctttaagatacccactaacagcaaccattttctcatcacagaggcaaggctaactgacctggaaaggaggagcctgggctgctgtccctcaacggaagaaccaaagagaaagaaaaaaacaaacagcacccgggaagcctggatcgtacctgatgtgcctcgctgtctgagagttcacttatttatttatctatgtatttatttatttattaattccattgcccagatgttgttatgtttattttgatatttaaagatatgcattcgctaattcactgtaatatcttaaaaggtcattttaatatgttaaagtttattttaataatgtttaatgtgttcaattaaagttatttaacttatatagttggaaggtgataaatttttaaacctatttattcattagtttctggggagagggtgagttgggaactagctacatcccacccacacagtgaaagagactggggataaggggtgggggtggggacaaatagatgcagtcggatggctttcatggaaggagtgtgcatgttcacatcatttttttgtaagcaccgaggagagtagaacagctgttatttaggtttcagtgtttgtaaactgtatgtacaacatttttgatgctggatttcaatgtaatgttgtgagtaacccttggacattttatgtcttcctcgtaaggcacagtgccttgcttagcaattgttttgtcatgccttttcgtgtcttgaagtggacacatttatttattcatgtatttttacaaataacaaaaaataaaaacgtctgtt

SEQ ID NO: 5 - DNA, 1218 bp, Human
cgggatcgatctggagctccgggaatttccctggcccgggactccgggctttccagccccaaccatgcataaaaggggttcgccgttctcggagagccacagagcccgggccacaggcagctccttgccagctctcctcctcgcacagccgctcgaaccgcctgctgagccccatggcccgcgccacgctctccgccgcccccagcaatccccggctcctgcgggtggcgctgctgctcctgctcctggtggccgccagccggcgcgcagcaggagcgcccctggccactgaactgcgctgccagtgcttgcagaccctgcagggaattcacctcaagaacatccaaagtgtgaaggtgaagtcccccggaccccactgcgcccaaaccgaagtcatagccacactcaagaatgggcagaaagcttgtctcaaccccgcatcgcccatggttaagaaaatcatcgaaaagatgctgaaaaatggcaaatccaactgaccagaaggaaggaggaagcttattggtggctgttcctgaaggaggccctgcccttacaggaacagaagaggaaagagagacacagctgcagaggccacctggattgcgcctaatgtgtttgagcatcacttaggagaagtcttctatttatttatttatttatttatttgtttgttttagaagattctatgttaatattttatgtgtaaaataaggttatgattgaatctacttgcacactctcccattatatttattgtttattttaggtcaaacccaagttagttcaatcctgattcatatttaatttgaagatagaaggtttgcagatattctctagtcatttgttaatatttcttcgtgatgacatatcacatgtcagccactgtgatagaggctgaggaatccaagaaaatggccagtgagatcaatgtgacggcagggaaatgtatgtgtgtctattttgtaactgtaaagatgaatgtcagttgttatttattgaaatgatttcacagtgtgtggtcaacatttctcatgttgaagctttaagaactaaaatgttctaaatatcccttggacattttatgtctttcttgtaaggcatactgccttgtttaatgttaattatgcagtgtttccctctgtgttagagcagagaggtttcgatatttattgatgttttcacaaagaacaggaaaataaaatatttaaaaatataaaa

SEQ ID NO: 6 - DNA, 1109 bp, Mouse
agactgcgcctcagtgcctccaacaagcttcctcgggcactccagactccagccacacttcagcctagcgccatggcccctcccacctgccggctcctcagtgctgcactggtcctgctgctgctgctggccaccaaccaccaggctacaggggctgttgtggccagtgaactgcgctgtcaatgcctgaagaccctgccaagggttgacttcaagaacatccagagcttgagtgtgacgcccccaggaccccactgcgcccagacagaagtcatagccactctcaagggcggtcaaaaagtttgccttgaccctgaagcccccctggttcagaaaatcatccaaaagatactgaacaaaggcaaggctaactgacctggaaaggaggagcctgggctgctgtccctcaacggaagaaccaaagagaaagaaaaaaacaaacagcacccgggaagcctggatcgtacctgatgtgcctcgctgtctgagagttcacttatttatttatctatgtatttatttatttattaattccattgcccagatgttgttatgtttattatgatatttaaagatatgcattcgctaattcactgtaatatcttaaaaggtcattttaatatgttaaagtttattttaataatgtttaatgtgttcaattaaagttatttaacttatatagttggaaggtgatacatttttaaacctatttattcattagtttctggggagagggtgagttgggaactagctacatcccacccacacagtgaaagagactggggataaggggtgggggtggggacaaatagatgcagtcggatggctttcatggaagtagtgtgcatgttcacatcatttttttgtaagcaccgaggagagtagaacagctgttatttaggtttcagtgtttgtaaactgtatgtacaacatttttgatgctggatttcaatgtaatgttgtgagtaacccttggacattttatgtcttcctcgtaaggcacagtgccttgcttagcaattgttttgtcatgccttttcgtgtcttgaagtggacacatttatttattcatgtatttttacaaataacaaaaaataaaaacgtctgtt

SEQ ID NO: 7 - protein, 360 aa, Human
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 8 - protein, 359 aa, Mouse
MGEFKVDKFNIEDFFSGDLDIFNYSSGMPSILPDAVPCHSENLEINSYAVVVIYVLVTLLSLVGNSLVMLVILYNRSTCSVTDVYLLNLAIADLFFALTLPVWAASKVNGWTFGSTLCKIFSYVKEVTFYSSVLLLACISMDRYLAIVHATSTLIQKRHLVKFVCIAMWLLSVILALPILILRNPVKVNLSTLVCYEDVGNNTSRLRVVLRILPQTFGFLVPLLIMLFCYGFTLRTLFKAHMGQKHRAMRVIFAVVLVFLLCWLPYNLVLFTDTLMRTKLIKETCERRDDIDKALNATEILGFLHSCLNPIIYAFIGQKFRHGLLKIMATYGLVSKEFLAKEGRPSFVSSSSANTSTTL

SEQ ID NO: 9 - DNA, 11250 bp, Human
aggaagtgcaggttcaaaacattcagagacagaaggtggatagacaaatctccaccttcagactggtaggctcctccagaagccatcagacaggaagatgtgaaaatccccagcactcatcccagaatcactaagtggcacctgtcctgggccaaagtcccaggacagacctcattgttcctctgtgggaatacctccccaggagggcatcctggatttcccccttgcaacccaggtcagaagtttcatcgtcaaggttgtttcatcttttttttcctgtctaacagctctgactaccacccaaccttgaggcacagtgaagacatcggtggccactccaataacagcaggtaggtgacttgttcagtctgagatgctggcaacatatttctaatggaagagatctggaaacttcagcctcctctgaggcccctgagaactggggtgatgaagtgtagaagcttagagctaaagacaactaagtcttcacctagacagtgttgcacaaaagaattttctgcaataatagaaatgtcctagacctgccctgcccagtatggtagccactagtcacacgtggccacggagcacttgaaatgtgactagtgtgactgcggatctcaatttttatttcattttattttattttattttttcttttttgagacaggctctcactccaatgcccaggctggagtgctgtggcatgatcttggctcattgcaacctcaacctacaggtctaggtgattctcccacctcagcctcccaagtagctgcagctacaggcatgtgccgctacactcagctaattttttgggttttgtttgtttgtttgttttgtttttgtttttgtaaagacagggtttcgtcatgttgcccaggctgatctcaaactcctgggctcaagcgatccgcccacctcggcctcccaaagtgctgggattacaggcatgggccaccttgcctggcctttattttaattaattttaatttaaatagctacacatggctagtagctagcatgctgaacagaatagaattagaagaagctcccgttttccaaaggcacacaacaaggcccagaaagaatgacttctccaagcaggattagaagccaggtttttgttttgtttttgtttgagacagggttttgctctgtcgcccaggctggaatgcaatggcacaatctcggctcactgcaacttctgcctcccgagttcaagcaattcttatgcctcagcctctggagtagctgggattacaggcgtgcgctgccacgcctggctaatttttgcatttttaatagaggcggggtttcaccatgttggccaggctgttctgtaactcctggcctcgagtgatccactcacctcagcctcccaaagtgctgggattacaggcatgagccaccgcaccagaagccagttttttaattcactccaccctgcatataaccgcagtgatctccatggaaatgaaattgacctaaggagcatgaagaaggaggctgactgggaaatgtcaaatctgggctcccctctctacatcctttgtatctgactttctgggacacagggaggtacctaatggagctcagaaactttaggctgggatcctgctccttattacagtgaacaggagataggatttgagagcagatttttagaagttttttaaactgaaaacctagttcagctgtgtcactctaggtatgttgcttaacctctcagagtcttcatttcctcttccagaaagtgcctgcttcgtgaggttatcctgaggattaattgatacaaattctggaaagcactttgcccagaagctagaacctggtagacactcaacattacagctaatatgacaattaatatccaggtcaaagtaaattagatgtcatcaaagtcagccttgaaagcagacccactgccgactgctttcccttgactggggatgtagtgagagaggcagcatgttatcagtgaataaactattccagtaataatgatcacaacaataaaggttaacattgtactttatgtatgttttacgtgatttacaagcaatccctcatttcatcctcaccacaaccccatgagttggattctgtattggccccattttacagatgagtaaactgagactcagaaaggaaaagtcacttgcccagaattgctcagctagccctggtgtcatctgcctctctccatatgtaataagcctctatctggttttagcatcaactggccaggtggcattgcattcttccccagcttgggaaagagaggtgacaaagactaagaaacatttgctgaacagttggactcaaagattctttctttggggtaggcaggcaatttaagtgcgtctgccacagggattgagtcccctctgggccctgggatgcatggagcaagagatagggtaaggaataatggaggggattgaggtgttgttcactggggtgggcacttgtgagacagcagaattagtgttcatggctctgcctggattaattcattcatttgtaatcaatagcaaattgataaaatacattcttagggtcacttaacattgtttttaaaaatgcatttggactttggtggaatcttcccttttaccaaaaggctttctttccaaattagagggtcttggctccaagggtggtaagctgctgcagagttggtgtcctcagacccaatgccaagtgtgcgaaacaagaaagaatatcaatacccagagatggacttgtggtcagagcagatggttggggagtgaggggaaagacaagaggcaggagtggggccaagaagtagaggcatcaggtccagggaggaacgccaccagactcaccagaagctttagtccatctctgtcattttaatgcactgctccctttgacatgtgaaagacattgaaggctatagtactgccaatggtatgatggggaagcctgaggcagctggaatgccaggagactcagacaaggcagctgaaggagccagcggctctgagatacagcaaagatgaaagaaatccatgcaatcctgggcaagccatttcctttgggcttcagtttccccatctgtaaaatggaaagactaggtagaaaacccctcaggtcccttcccaatataaactctacgagtctatgaaatgactatgcatccgagagctgatctgggaactaattattcaaagttatcaaaatcacccgcaccctcaggagctccacccctgaattgcattgagcaatctaatctaccactcacttcctccactgtgttctcaccaccacctccctttcataggtcacagctgctcttctggaggtgtcctacaggtgaaaagcccagcgacccagtaagtcttggaacacccacccttcccaccagctagaatgtctctgttctctgtttcccttgatacccagcctacatttatcctaagcatctctccccacccttggtgggcccctcaccctcaccccttggcctcttcattttctccctttggggctctccatccccaaaggaagaacttctgttcctcctccctcacaaccctctccaaccactcagggccacttgctgcccatgcttttcccttggcctggcagccttccaagactcagccagccctcctggcctgggctaggactgggtctgaggatgggccgaacactgttcgcttcccacttcccgacactaaatgcaatatttgtggattatatttatagagagaaagaatactggaactgtactaaaatgactcttttgttgttaaaaagattgccttagatgtttgagatcttgtaaatgtctctcgttctatagcattttcatgtaataaattagaattcatgccatcatctatggagtttccaaactaccatgagtcactaaggggctggcattttccctaagtcaaggaagctccctttttatcctggaatatatgactgtgtggtcagccggacccagccagttcaagggtgtttggtttatcatccctgcaagagctgtttctttcattctgggccaaagaaacggcaaaagaaaacaatgcataaaacatgaaagaatgtgagtggaccatatacaggttgatttaatgatatctcaagaacttagctttgctgggcacggtggcttacacctgtaatctcagcactttgggaggctaaggtgggcggatcacctgaggtcaggagttcgagaccagcccgaccaacatagtgaaaccccgtctctactaaaatacaaaaattggccgggtgcagtggcaggcgcctgtaatctcagctacttgggaagctgaggcaggagaatcacttgaacccgggaggtggaggttgcagtgagccgagatcacgccattgcactccagcctggcaacagagcgagactgcatcaaaaaaagaaaaaaagaaagaaaaagagaacttagcttacaaatatgtttaatagatgaggtgcactttttttaagaggaagggctatagactacaaataaggcaagtgtagtcctttatacctcactggaacagtggtttcaccacttctccaccctgtgatcgcaggctggttactgagcctccctgttaggtcagctgatcctccttgtgcctcagtcttctagaaacagggcctaattgtatctatttgtaatgagccatggagatcatgtacctgtgaggcacaaagcagacctcagatgtgtcatctccttcccttagtttgtccctctaactgccaagtcatcctctgtagagattggtaaactaaaaaacaacaccaacaacaaacacccctctccctaccctaggagctggaggtcactggcctgacctggttctccttgctgagttctctaccttctgtcctgacttgcatgctctccctctggaacctgaagctgacccataatgcctgggagcatgagctcacccatacacctccagcccacccccagcacagctgagcactggattatccacagtgaggatttacctgtgaaacatgctctgccactgctgcctgtgctgacatggcagtttctgcagcagcctcagtcctggcttctcacagccaatattcatacatacaaggccctgctctctaagagcacaagctctctgcacccaatatgccctacatctactctcaaagatgggtcttctgttgggtcatttgttcccttaagagcatctgtaatcatctgctcccagaagtgatccacccttgacctcttgacttcattggacataccctgggcctctcagcaaacttgctctcaaccatgggactgaggagctggtgacttctttcaggtcttgctgaccatactggtccagcaagctgtattcccagccctctcaatacctccccagccttctggccaggcagggccagaaatgagggattgcttataaattacgtaagacatacataaagtacaatgttaacttttttttttttttttttgagacggagtctcctctgtcgcccaggctggagtgcagtggcgccatctcggctcactgcaagcttcgcttcccgggttcacaccgttctcctgcctcagcctcccgagtagctgggaccacaggcgcccgccaccacgcccagctaattttttgtatttttagtagagacggggtttcactgcattagccaggatggtctcgatctcctgacctcatgatccgcccatctcggcctcccaaagtgctgggagccaccacgcccggcccaaatgttaacttttattgttgtgatctagtatagtacattacaactgattgaatcttctccatcagttgcctcggcatgctgggaaggcaatgtatctggcccctaactcttaaaagagaaactaaaaacttgtcaggaaggtgatggctaagaattccatcactcttgggggaaaagtggcattatttttccagttttgcttaatcttcttcagctttcttctttgctacatatgattgatgacaacagagacttcctttctgctaaaaagaaatcctccctgggacttttgttgttatttttgtaatatttgtatttgctttgcaaggccgtgtaccctttgattaatcttttttttaattaagacataagtcacataccccaaaagtcacccatttaaagtttttagcatattctacaaccattgccactaattccagaacacttttatcactcaaaagaaaaactccgtagtcattaacattcctctcccagctccccttgaccctggaaaccaccaatctactttctgtctctaccaattctacccatttgcctattttggtcatttcacatatatggaatcacacaataagaaaacttttgtgtctgccttttttcacctagactaacattttcaaggttcatccatgttgtattgtgtatcagtacctcattccttctcatggctgaataatattctgttgtacataatacagtcataagttgcttaacaatgcggatgcattctgagaaatatgtcatgtgaacatggtagagtatacgatgctatagcctactacacacatgggctatatggtagagtactgctcctaggctacaaacctactcagcatgttactgtactaaatactgtaggtaattataacacaatggtattcttatatataaacataggaaaagaacactaaaaatacagtattataatcttatgggaccactgtggtatttgtgatgtgtcattgaccaaaaagtctttcagttgctcatgactgttcctcaatttgtctgtccgttcatcagttgatggatatttggtttgtttccactttttggccttaatgatgaatgctactatgaacattagctttcaattctcttgggtgtatacctaggttggggtttgctggttcatgtggtaactctatgtttaacttttttgagcaactgctaagctgttttccaaagcagctgcaccatcttacattcccacctgcaatgtgggagagttctgacttttccacttcctcacccacattgttcttgggcatctttttctgagtgtgcccagcctggtgagtgtgaagtgggttctgggacctttaacttcctggtttttctctcacactgggttccattttgggggtagattacggacttggacatggttggtggtagggtggtcatcccttactctaaggcaatggccggagggtgatctattttgtggttcagaaagaagctaattagggagctggattgctttagttgactgctttgagaaactgggatttttttttttttttttttttttttttgaggcagagtctcgctctgttgcccaggctggagtgcagtgatgtgatttcggctcactgcaacctccacctcctgggttcaagcaattcttgtgcctcagcctcccaagtagctgggattacaggtgcacgtcaccatacctggctaatttttgtattattagtagagacagtgtttcaccatgttagccaggctgctctcaaactcctgacctcaagtgatctgccacctcagcctcccaaagtgctgggattacaggtgtgagccaccactgggataatatttttgaataactgcagcccaccctccacacccaggcttccccagagcaggatctgtgctgtccctgtggcaagggcagagccccaggagctagactaaatctgagaggaacaggggagtcaaggctagatggaggcacacacatgcaggacaggaagggtctccagcagggctggaaagccaccaagcaagggtagtgcaaaccctgtcttctctgcttcctgctgcccctctcttattgggtagcagactggctgtgtctgcttatcccgcatggtggcacatggctgcccagagctccccaaataacctgttacgtttccacccacagggagaatcaggcagcccgcttgcttattgtttttaatatacttttccaaactacacagacattcccaaagcgggtctccttctacccaaaagagaaacgctgggccttactaattaactgaagactctgctagctcgagccttccaaaactccatgccacaattgttcaaaccattttccagaatacatcttttaaaataacttttagaaaattgaacaaagggattcatgtctcagctttatagtcaaaccatgatcttttctgagggtatagcccatttggagttcctgcttaatcccctgattaaaaactgaatggggctgagtgcagtggctcatgcctatattcccagcactttgggagacagaggcaggagaattacttaaggtcaggagtttaagaccatcctgagcaacataacaagtccccatctctaagacaaaaaaaaaagaactgagtgacatctcacatctcacatttctgaacattaaacccagccttgatagccaaagatgctcgccactgaaggatccaggtagtattgagggttctgtggggattatccaaagagaactttctacaaagttttaggtgatggcgatgctaaaagaaatgctaagaatttctctcttatattaaagagaactatggtcctctcataaaatgtaccatttatcaccaaatttatctcataacctaagagctaccacttacaaatttgaagggaaaaattactacattgtaatactcaagccaacacaaagaatcctatcccagtttcttgagtggatgggcaagaatatggggaatttattatgcagtaaccttcatctctcttctataggtcaggatttaagtttacctcaaaaatggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaagacctcctgcctaagtgcagccccgtggggttcctcccttctcttcacagtcacattccaagcctcatgtccactggttcttcttggtctcagtgtcaatgcagcccccattgtggtcacaggaagtagaggaggccacgttcttactagtttcccttgcatggtttagaaagcttgccctggtgcctcaccccttgccataattactatgtcatttgctggagctctgcccatcctgcccctgagcccatggcactctatgttctaagaagtgaaaatctacactccagtgagacagctctgcatactcattaggatggctagtatcaaaagaaagaaaatcaggctggccaacggggtgaaaccctgtctctactaaaaatacaaaaaaaaaaaaaaattagccgggcgtggtggtgagtgcctgtaatcacagctacttgggaggctgagatgggagaatcacttgaacccgggaggcagaggttgcagtgagccgagattgtgcccctgcactccagcctgagcgacagtgagactctgtctcagtccatgaagatgtagaggagaaactggaactctcgagcgttgctgggggggattgtaaaatggtgtgaccactgcagaagacagtatggcagctttcctcaaaacttcagacatagaattaacacatgatcctgcaattccacttataggaattgacccacaagaaatgaaagcagggacttgaacccatatttgtacaccaatattcatagcagcttattcacaagacccaaaaggcagaagcaacccaaatgttcatcaatgaatgaatgaatggctaagcaaaatgtgatatgtacctaacgaagtatccttcagcctgaaagaggaatgaagtactcatacatgttacaacacggacgaaccttgaaaactttatgctaagtgaaataagccagacatcaacagataaatagtttatgattccacctacatgaggtactgagagtgaacaaatttacagagacagaaagcagaacagtgattaccagggactgaggggaggggagcatgggaagtgacggtttaatgggcacagggtttatgtttaggatgttgaaaaagttctgcagataaacagtagtgatagttgtaccgcaatgtgacttaatgccactaaattgacacttaaaaatggtttaaatggtcaattttgttatgtatattttatatcaatttaaaaaaaaacctgagccccaaaaggtattttaatcaccaaggctgattaaaccaaggctagaaccacctgcctatattttttgttaaatgatttcattcaatatcttttttttaataaaccatttttacttgggtgtttata

SEQ ID NO: 10 - DNA, 7256 bp, mouse
gtcagtagtttcctcatcacggctgcctcactttcttccagttcaaccagccctgacagctcccaagccttgagtcacagagagttgggagccactctgctcacaaacagcgtcgtagaactactgcaggtaggagagctgattggtcacggactagggaatatatttctggtaaaagggaccagggaactctgcctttcctggagccctgggaagcggcatgacgcattgtagaacctcagagctgaggtctgcatcttcagatagctggtgctatatcaaagcactttttataataagcatgctctatatctgcatggtctaatatggaagtcactggtcacctgtggccactgagcccttgaaatgtagcagcgacagccactttagtaaatttatttgtttgtttgtttgtttgtttttgtgtgtatgtgtgtgtgccgtgggatactagtgaaggtcagaggatagtttcatggagtcagttcttcacctgggatctgaggatccaacccaggtcacctgatgaacagcaagtgcttttgccaacaaaaccatctccccagcttccaatgaagtgaattctagtctatctcagctgcttttcatttagagagttaagtggcctgaagctacaattctgggcaggaaagacctgaaacaaaccagtgtcttccaggagagcaacaaggtccagagagagacggtgcctttaccctgacccccgagggcaggaccgagattagaactcttttttttttttaaagaaacaaaactatgcggcataggctacataataagacaaaacaaaatttgaaagaaagaaagaaagaaagaaagaaagaaagaaagaaagaaagaaagaaagaaagaaaagcagaagtgggtgattgagaaatgcccaatctgggttcatttcccatcccagtggtattttgtctgggacagagcagttacctatggagctaggaaacctgaggctggctgaggccaggaccctgctcctcccagcatttgataaatgaaaacataagagcagactttaatatccattttaaaacgtcgaccgcccagttcagctgcgtctctcaaggtaagttgcctaacccccagggactccgtttcctcatccagaacatggcgacttcattgatgtgagggttgaaagacaaaatagatggtagatcccctttgcctgtgaggcggcaagccagtcagccatccagattacaaccagatgacaactaataagacagccattatccaggtcagagcatgtgaaggtgcctacaggagttggccctgaaaactaggtaggctttcttctctccctgactcattctcagtctggggatgtgatgggagaggccagtcacctgtagatacactacttcagttctaagaatcacagtataagctatctctgtggtatgtgtgtgcatgtgtatgtgtgtgacatgtagctaccacacaacccatgggttgtgatcctttctataaaaatctcatcgggtcacttcccagtacgaactctctaaatccataagatgactccatccgtgggcggtgggctgagaattcatacttaaaacatcaaactcatctcacgccctcgggagctcctccccctcttgagcaatctaacccatccctcacttcctcttaagaatttcactgctcttcatgggtcacagctgcctaccttaggtgtcccacaggtgaaaagccagcatcctggtgagtctggaacacctcctaccaccacctagaatcttctctctgtgtcactcaacacaggacctgaccatgagtcatcatgcccctccttcctggcttgactcctaaacattatgcctccttagcacagaggaagggcttctgacctcctttctgtcacctggcaaaaactgcttagaactacttgcaattcagcccctacacttggcctgccatctttccaagtctgcctaccctccttgcctaggctgcagttgggtctggagatagatgtaccaggcactgaggactctcaaacctgtggggcatttatgggctgtatttctaaagagaatgagaattggggtcagggtacaatgacttagttttctgttgttgaaatgtttcccagccctggagcttctggactaatgcaaattccaaggagttggtacttccattcaatccaggagctcctacttcatcacagaggacatgacagggccctcagttgaagatgtttcctttgccatcccagcaatagctatttggttcatttgcagcctaagggatagcaaaaagatggtggtgtccaagcttgcatgcttgactgaaaggtgattcaagatcttagatctatggggctgtgactctgtggcagagcgggtacttagcgtgtgcagggccaatcttcgggaagcatgataaagcgaaatgcttaggagtgagcatatctaatataacagggacgatcctccatcagtaaactaaaggcagcgcgtggcgtggacaaactaatgacgtgaaatcccgctgcaccacaacagccctgtggtcgcagactgtttactgggccttctgggtctcatcgtccgtcccgccagcaatgagctaattatagcacttgtgatgactgagggaaatcttgcacaaagcagatctcaactgtcatccattccttccctctgtgccttcagccatgaatgcattcctttcagagattagtatgttaaaatgcaaacaccaactcttgtcccctcgcctgtggctgtgcatttgctggtccctcctggccctctcctcagagtccaccttccatcctggcctgcatggtctccctgtagaacactgaacctgcctttgctgcctggacagccctcacagtcctgcattttatctgaattccacaatctagctacctgccgaattcaagtgcacaagttaggggagcaagcttttatccaaccagccactttcctaacttcattgttttggttttcaaatgtgttgttgttgttattgttgttgttttattgttattgctgctgatgatggtgatggtgatgttttgaatgaggtctcatataggtcaagatagacttgaactgctttgtagctaaagatgactttaaagtcctgatcttccttcctctacccccagtatactgagtgcggggattgcctgcagatgccatcatacctcgtttattcagttctgggagccctacctaggactttgagaatctcaggcaagcattctatcacctgagctccacctctgcccatttttgtcattttctttacaattatttttaactttttatgtacatgggtgtttcacctgcaagtatgtaggtttaccctgtgcatgcctggtacctgagggggacagaagatcccttggaactggggttcctgggagttgttagctgctgggtgtgtgttgagaaaccaagcctgagtacactacaagtgcagccaatgctcataaccactaagtcatctctccagccccattttttttgttattattattattattgttttaatatttgtatttgtttgtaagattgtacacctttttaatgttttactagcatattttagctatgcatatgagtttaattataaaaatttcatatatatgtatatatacatatttacatatgtgtatgtatacatgcatatttatatgtgtgtacatatatacatatttatatatgtatatgtgtatatatactgtgtatagcatatatgatatattatatacacatatataaatcatatataaacatatgttttttttgaaacagggtcttagttgtcctcaaacatgctatgtagccaaggatgtacaaattatgtatatatcatatatacaaattatatctatacatatatagaagcagagttgtatatatgtatataatatattcaaattatatatgtcatatacaaatatttgtatatatgtatattatatatacaaattatatatatataaattatatatacatatgcatatataaatgtgtgcatgtataaaatttgtacagccttggctactagcatgtttgagcccaaccaagtccctgtttcaaaaaacaatcttgaagagaactgtagtttttatgccccacaaatatctcacttattatttattactattttgcctcaaatgtttactgctatcatttacaaacttgaaggaaatagtgtcttccttgcaatatacaagccaatggaaaaatcctcacgccccagtttcttggggggggcggggtaagacaagaatcagggtggctttgccgtgcaataaccttcatctctcctctattgacaggattaagtttacctcaaagatgggagaattcaaggtggataagttcaacattgaagatttcttcagtggagatcttgatattttcaattatagctctggcatgccctctattctgccagatgctgtcccatgccactcagagaacctggaaatcaacagttatgctgtggttgtaatatacgtcctggtgactctgctgagccttgtggggaactccttggtgatgctggtcatcttatacaaccggagcacctgctctgtcaccgatgtctacctgctgaacctggccattgctgacctgttctttgccctgaccttgcctgtctgggctgcatctaaagtaaatggatggacttttggctcaaccctgtgcaagatattctcatacgtgaaggaggttaccttctacagcagtgttctgctactagcctgcatcagcatggaccgctacctggccattgtacatgccacaagtacactgatccagaagagacacttggtcaagtttgtgtgcatagccatgtggttactatcagtaattctggccctgcccatcttaattctacgaaatcctgttaaggtaaacctttctaccttagtctgctatgaggatgtaggtaacaatacatcccgtttgagggtcgtactgcgtatcctgcctcagacttttggcttcctcgtgccgctgctcatcatgctgttctgctacgggttcacactgcgcaccctctttaaggcccacatggggcagaagcaccgggccatgcgggtcatcttcgctgtcgtccttgtcttcctgctctgctggctgccctacaacctggttctgttcacagacaccctcatgagaaccaagctgatcaaggagacctgtgagcgccgcgatgacattgacaaggccttgaatgctacggagattcttggcttcctccacagctgccttaaccccatcatctatgcctttattggccagaaatttcgccatggacttctcaagatcatggctacttatggccttgtcagcaaggagttcttagccaaggagggaaggccttcttttgttagctcgtcttcagcaaacacctctactaccctctaaagactgttcacctaaacggtggcccctcggggttccttcttgtctttcagcatggctcattaccagagactgtggtatttgaattgatgcagcccctcctctacaattacagggagaaagaggtcacgttcttagcagagcccccagagtttagaaccccctatattggctgtctgctcccttccatcttggtatgcctactgatagagttgatccatcctaacactagaccccaaacactcttttctaagaagcacacgttacaattacagtgagatactgcctcctacccatcagaacagttagcagtaaaaggaagaggtggaggagagaaaatggcaagtgacaatgaggaagtagaaaaagggaactcttcaccttaccagtagacgagtaccagagtcccctcacacaggaacatagcatagcagttttccatttaaaaaaaaattaagggctggtgagatggctcagtgggtaagagcacccgactgctcttccaaaggtccgaagttcaaatcccagcaaccacatggtggctcacaaccatctgtaacgagatctgatgccctcttctggagtgtctgaagacagctacagtgtacttacatataataaataaataaatctttaaaaaaaaaaattaaatatagaattggcatgtaatccagcaagtccatctctaagtagggtacctaaaaaaattgaaagccagggttcagacatttgtatgctgatactcacaacagcattattcatcatgaaaatgatggccaacagatgaatgggtaagttaaacaaggtgtctctacacaacaaaagagcgtccacaatgagaagtgccaaaaataatcaaatagtgttgactggaggctgggtgaggaagggtggggagttcgtgtagaagacacagctccagttagggatgatgggaaagccctgaaggtgggccctgatccctgtgtagtagtgtgaatgcactcattaccactgtaacaaaagtgttaaaatgaaagctgagtattgctccctgggagagtgcttatttaacatttctaagaactcaggctcaaatcctagcacctcgacaaacaaacaaacaaacaaacaaaaaagctaaaatggttatttttgttatgtgtgttttacctcactttaccaaaaatataaaaatctaagtctaaaaaatgttcaccatcccataaacaaccaccaaacccagacactaggcagatgccaacaagaacctgctgacaggaggctgatatagctgtctcctgaggagctctgccagtgcctggaaaatacagaagtggatgctcacaatcatccattggatagaggacaaggtcccaaatgaagaagctagcgaaggtatccaaggagctaaaggggtctgaagccccagaggaggaacatcagtatgaactagccagtacccccccagagctccttggaactaaaccaccaatcaaagaaaacacatggtggaacttgtggctctagctgtatatgtagcagaggatggcctagtcagtcatcaatgggaggagaggcccttggtcctgtgaaggctctatgcctcaatataggggaatgccaggaccaggaatgggagtaggtggattggggagcagggggagagggaagggaataggggatatttggaggggaaactaggaaaggggataacatttgaaatgtaaataaagaaaatatctaataaaaaaaatgttctttaaccactgaaccaatgcagaactatctaccttataatttcttgtaaagtgtgactattaaatgtcgttttaaaaaata

SEQ ID NO: 11 - DNA, 2879 bp, Human
aggaagtgcaggttcaaaacattcagagacagaaggtggatagacaaatctccaccttcagactggtaggctcctccagaagccatcagacaggaagatgtgaaaatccccagcactcatcccagaatcactaagtggcacctgtcctgggccaaagtcccaggacagacctcattgttcctctgtgggaatacctccccaggagggcatcctggatttcccccttgcaacccaggtcagaagtttcatcgtcaaggttgtttcatcttttttttcctgtctaacagctctgactaccacccaaccttgaggcacagtgaagacatcggtggccactccaataacagcaggtcacagctgctcttctggaggtgtcctacaggtgaaaagcccagcgacccagtcaggatttaagtttacctcaaaaatggaagattttaacatggagagtgacagctttgaagatttctggaaaggtgaagatcttagtaattacagttacagctctaccctgcccccttttctactagatgccgccccatgtgaaccagaatccctggaaatcaacaagtattttgtggtcattatctatgccctggtattcctgctgagcctgctgggaaactccctcgtgatgctggtcatcttatacagcagggtcggccgctccgtcactgatgtctacctgctgaacctagccttggccgacctactctttgccctgaccttgcccatctgggccgcctccaaggtgaatggctggatttttggcacattcctgtgcaaggtggtctcactcctgaaggaagtcaacttctatagtggcatcctgctactggcctgcatcagtgtggaccgttacctggccattgtccatgccacacgcacactgacccagaagcgctacttggtcaaattcatatgtctcagcatctggggtctgtccttgctcctggccctgcctgtcttacttttccgaaggaccgtctactcatccaatgttagcccagcctgctatgaggacatgggcaacaatacagcaaactggcggatgctgttacggatcctgccccagtcctttggcttcatcgtgccactgctgatcatgctgttctgctacggattcaccctgcgtacgctgtttaaggcccacatggggcagaagcaccgggccatgcgggtcatctttgctgtcgtcctcatcttcctgctctgctggctgccctacaacctggtcctgctggcagacaccctcatgaggacccaggtgatccaggagacctgtgagcgccgcaatcacatcgaccgggctctggatgccaccgagattctgggcatccttcacagctgcctcaaccccctcatctacgccttcattggccagaagtttcgccatggactcctcaagattctagctatacatggcttgatcagcaaggactccctgcccaaagacagcaggccttcctttgttggctcttcttcagggcacacttccactactctctaagacctcctgcctaagtgcagccccgtggggttcctcccttctcttcacagtcacattccaagcctcatgtccactggttcttcttggtctcagtgtcaatgcagcccccattgtggtcacaggaagtagaggaggccacgttcttactagtttcccttgcatggtttagaaagcttgccctggtgcctcaccccttgccataattactatgtcatttgctggagctctgcccatcctgcccctgagcccatggcactctatgttctaagaagtgaaaatctacactccagtgagacagctctgcatactcattaggatggctagtatcaaaagaaagaaaatcaggctggccaacggggtgaaaccctgtctctactaaaaatacaaaaaaaaaaaaaaattagccgggcgtggtggtgagtgcctgtaatcacagctacttgggaggctgagatgggagaatcacttgaacccgggaggcagaggttgcagtgagccgagattgtgcccctgcactccagcctgagcgacagtgagactctgtctcagtccatgaagatgtagaggagaaactggaactctcgagcgttgctgggggggattgtaaaatggtgtgaccactgcagaagacagtatggcagctttcctcaaaacttcagacatagaattaacacatgatcctgcaattccacttataggaattgacccacaagaaatgaaagcagggacttgaacccatatttgtacaccaatattcatagcagcttattcacaagacccaaaaggcagaagcaacccaaatgttcatcaatgaatgaatgaatggctaagcaaaatgtgatatgtacctaacgaagtatccttcagcctgaaagaggaatgaagtactcatacatgttacaacacggacgaaccttgaaaactttatgctaagtgaaataagccagacatcaacagataaatagtttatgattccacctacatgaggtactgagagtgaacaaatttacagagacagaaagcagaacagtgattaccagggactgaggggaggggagcatgggaagtgacggtttaatgggcacagggtttatgtttaggatgttgaaaaagttctgcagataaacagtagtgatagttgtaccgcaatgtgacttaatgccactaaattgacacttaaaaatggtttaaatggtcaattttgttatgtatattttatatcaatttaaaaaaaaacctgagccccaaaaggtattttaatcaccaaggctgattaaaccaaggctagaaccacctgcctatattttttgttaaatgatttcattcaatatcttttttttaataaaccatttttacttgggtgtttata

SEQ ID NO: 12 - DNA, 3046 bp, Mouse
gtcagtagtttcctcatcacggctgcctcactttcttccagttcaaccagccctgacagctcccaagccttgagtcacagagagttgggagccactctgctcacaaacagcgtcgtagaactactgcaggattaagtttacctcaaagatgggagaattcaaggtggataagttcaacattgaagatttcttcagtggagatcttgatattttcaattatagctctggcatgccctctattctgccagatgctgtcccatgccactcagagaacctggaaatcaacagttatgctgtggttgtaatatacgtcctggtgactctgctgagccttgtggggaactccttggtgatgctggtcatcttatacaaccggagcacctgctctgtcaccgatgtctacctgctgaacctggccattgctgacctgttctttgccctgaccttgcctgtctgggctgcatctaaagtaaatggatggacttttggctcaaccctgtgcaagatattctcatacgtgaaggaggttaccttctacagcagtgttctgctactagcctgcatcagcatggaccgctacctggccattgtacatgccacaagtacactgatccagaagagacacttggtcaagtttgtgtgcatagccatgtggttactatcagtaattctggccctgcccatcttaattctacgaaatcctgttaaggtaaacctttctaccttagtctgctatgaggatgtaggtaacaatacatcccgtttgagggtcgtactgcgtatcctgcctcagacttttggcttcctcgtgccgctgctcatcatgctgttctgctacgggttcacactgcgcaccctctttaaggcccacatggggcagaagcaccgggccatgcgggtcatcttcgctgtcgtccttgtcttcctgctctgctggctgccctacaacctggttctgttcacagacaccctcatgagaaccaagctgatcaaggagacctgtgagcgccgcgatgacattgacaaggccttgaatgctacggagattcttggcttcctccacagctgccttaaccccatcatctatgcctttattggccagaaatttcgccatggacttctcaagatcatggctacttatggccttgtcagcaaggagttcttagccaaggagggaaggccttcttttgttagctcgtcttcagcaaacacctctactaccctctaaagactgttcacctaaacggtggcccctcggggttccttcttgtctttcagcatggctcattaccagagactgtggtatttgaattgatgcagcccctcctctacaattacagggagaaagaggtcacgttcttagcagagcccccagagtttagaaccccctatattggctgtctgctcccttccatcttggtatgcctactgatagagttgatccatcctaacactagaccccaaacactcttttctaagaagcacacgttacaattacagtgagatactgcctcctacccatcagaacagttagcagtaaaaggaagaggtggaggagagaaaatggcaagtgacaatgaggaagtagaaaaagggaactcttcaccttaccagtagacgagtaccagagtcccctcacacaggaacatagcatagcagttttccatttaaaaaaaaattaagggctggtgagatggctcagtgggtaagagcacccgactgctcttccaaaggtccgaagttcaaatcccagcaaccacatggtggctcacaaccatctgtaacgagatctgatgccctcttctggagtgtctgaagacagctacagtgtacttacatataataaataaataaatctttaaaaaaaaaaattaaatatagaattggcatgtaatccagcaagtccatctctaagtagggtacctaaaaaaattgaaagccagggttcagacatttgtatgctgatactcacaacagcattattcatcatgaaaatgatggccaacagatgaatgggtaagttaaacaaggtgtctctacacaacaaaagagcgtccacaatgagaagtgccaaaaataatcaaatagtgttgactggaggctgggtgaggaagggtggggagttcgtgtagaagacacagctccagttagggatgatgggaaagccctgaaggtgggccctgatccctgtgtagtagtgtgaatgcactcattaccactgtaacaaaagtgttaaaatgaaagctgagtattgctccctgggagagtgcttatttaacatttctaagaactcaggctcaaatcctagcacctcgacaaacaaacaaacaaacaaacaaaaaagctaaaatggttatttttgttatgtgtgttttacctcactttaccaaaaatataaaaatctaagtctaaaaaatgttcaccatcccataaacaaccaccaaacccagacactaggcagatgccaacaagaacctgctgacaggaggctgatatagctgtctcctgaggagctctgccagtgcctggaaaatacagaagtggatgctcacaatcatccattggatagaggacaaggtcccaaatgaagaagctagcgaaggtatccaaggagctaaaggggtctgaagccccagaggaggaacatcagtatgaactagccagtacccccccagagctccttggaactaaaccaccaatcaaagaaaacacatggtggaacttgtggctctagctgtatatgtagcagaggatggcctagtcagtcatcaatgggaggagaggcccttggtcctgtgaaggctctatgcctcaatataggggaatgccaggaccaggaatgggagtaggtggattggggagcagggggagagggaagggaataggggatatttggaggggaaactaggaaaggggataacatttgaaatgtaaataaagaaaatatctaataaaaaaaatgttctttaaccactgaaccaatgcagaactatctaccttataatttcttgtaaagtgtgactattaaatgtcgttttaaaaaata

(19) United States
(12) Patent Application Publication
(10) Pub. No.: US 2021/0163610 A1
(43) Pub. Date: Jun. 3, 2021

(54) REFRACTORY ASTHMA PROPHYLACTIC/THERAPEUTIC AGENT SCREENING METHOD, AND REFRACTORY ASTHMA PROPHYLACTIC/THERAPEUTIC AGENT

(71) Applicant: THE UNIVERSITY OF TOKYO, TOKYO (JP)

(72) Inventors: TETSU AKIYAMA, TOKYO (JP); YUSUKE YAMAZUMI, TOKYO (JP); OH SASAKI, TOKYO (KR); HIROAKI HARADA, TOKYO (JP)

(21) Appl. No.: 17/050,894

(22) PCT Filed: Apr. 18, 2019
(86) PCT No.: PCT/JP2019/016695

§ 371 Date: Oct. 27, 2020

(30) Foreign Application Priority Data
Apr. 27, 2018 (JP) 2018086517

(51) Int. Cl. C07K 16/28; C07K 16/24; A61P 11/06; C12Q 1/6883; G01N 33/68; C12N 15/115

(52) U.S. Cl. CPC C07K 16/2866; C07K 16/24; A61P 11/06; C12Q 1/6883; A61K 2039/505; C12N 15/115; G01N 33/6893; C12N 2310/16; G01N 33/6863

(57) ABSTRACT

Provided are a refractory asthma prophylactic/therapeutic agent screening method, and a refractory asthma prophylactic/therapeutic agent. This refractory asthma prophylactic/therapeutic agent screening method uses at least one indicator selected from the group consisting of: inhibited activity of the CXCL2 protein or the CXCR2 protein; suppressed expression of the CXCL2 gene or the CXCR2 gene; and suppressed expression of the CXCL2 protein or the CXCR2 protein.

### TECHNICAL FIELD

The present invention relates to a method for screening prophylactic or therapeutic agents for refractory asthma and to a prophylactic or therapeutic agent for refractory asthma.

### BACKGROUND ART

Asthma mortality has been reduced markedly due to the widespread use of steroids. However, the refractory asthma patients, who poorly respond to steroids account approximately 5 to 10% of asthma. The number of deaths from refractory asthma makes up approximately 40% of the total number of deaths due to asthma, and the treatment of refractory asthma makes up about 40% of the asthma-related medical expenses. Therefore, the development of effective therapeutic agents for refractory asthma is an urgent and important issue. Refractory asthma is reported to be classified into several clinical phenotypes such as eosinophil-dominant refractory asthma and neutrophil-dominant refractory asthma. For eosinophil-dominant refractory asthma, molecularly targeted agents such as anti-IL-5 antibodies and anti-IgE antibodies have been developed and administered clinically. On the other hand, effective drugs for refractory asthma exhibiting neutrophilic infiltration have not been developed. For example, agents for inhibiting neutrophil elastases have been developed, however it has been reported that these agents have failed to show efficacy for refractory asthma.

CXCL1, CXCL2, and CXCL5 are the members of the inflammatory chemokine CXC subfamily. Inflammatory signals activate CXCL1, CXCL2, and CXCL5 secretion from various cells such as blood cells, fibroblasts, vascular endothelial cells, vascular smooth muscle cells, alveolar epithelial cells (see, for example, Non-Patent Documents 1 and 2). Patent Document 1 discloses an antibody that binds to various chemokines. CXCR2, a receptor for chemokines, is expressed on neutrophils and promotes migration of neutrophils through the interaction with its ligands, for instance with CXCL8 in humans and CXCL1 and CXCL2 in mice. It has been reported that CXCR2-mediated migration of neutrophils plays critical roles in the development of various diseases. In mouse models of inflammatory bowel disease, for example, administration of anti-CXCR2 antibodies has been shown to reduce the number of neutrophils in the intestinal mucosal layers and to alleviate the pathogenesis (see, for example, Non-Patent Document 3).

Patent Document 1: Japanese Patent No. 6105146

Non-Patent Document 1: Thorax. 2007 June; 62(6):475-82.

Non-Patent Document 2: Trends Immunol. 2011 October; 32(10):452-60.

Non-Patent Document 3: THE JOURNAL OF PHARMACOLOGY AND EXPERIMENTAL THERAPEUTICS: Vol. 329, No.1; 329:123-129,2009

### DISCLOSURE OF THE INVENTION

### Problems to be Solved by the Invention

It has been recently shown that in a subgroup of patients with refractory asthma, neutrophilic infiltration is activated in the lung, and there is increasing evidence that the accumulating neutrophils mediate the pathogenesis of refractory asthma. It has been reported that various molecules have been shown to involve in the infiltration of neutrophils, but unfortunately, responsible factors for the pathogenesis of refractory asthma are still unclear, and molecularly targeted drugs have not been developed yet.

It is an object of the present invention, which has been made in light of the above circumstances, to provide a method for screening prophylactic or therapeutic agents for refractory asthma and to provide a prophylactic or therapeutic agent for refractory asthma.

### Means for Solving the Problems

The present inventors have completed the present invention based on the findings that, in refractory asthma model mice, administration of an inhibitor of CXCL2 or CXCR2, which is a neutrophil migration factor, such as a monoclonal antibody against CXCL2 or CXCR2, can inhibit neutrophilic infiltration into the bronchial area and inflammation of the bronchial area. Specifically, the present invention is directed as follows.

A first aspect of the present invention is directed to a screening method including screening a prophylactic or therapeutic agent for refractory asthma using, as an indicator, at least one selected from the group consisting of inhibition of CXCL2 or CXCR2 protein activity, inhibition of CXCL2 or CXCR2 gene expression, and inhibition of CXCL2 or CXCR2 protein expression.

A second aspect of the present invention is directed to a prophylactic or therapeutic agent for refractory asthma, including an inhibitor of CXCL2 or CXCR2 protein activity, an inhibitor of CXCL2 or CXCR2 gene expression, or an inhibitor of CXCL2 or CXCR2 protein expression.

### Effects of the Invention

The method according to the first aspect enables the screening of prophylactic or therapeutic agents for refractory asthma. The method according to the first aspect also enables the screening of prophylactic or therapeutic agents with less side effects for refractory asthma since CXCL2 and CXCR2 are located at a downstream part of the signal transduction pathway and, among the immune cells, neutrophils can be selectively reduced. The agent according to the second aspect can prevent or treat refractory asthma. The prophylactic or therapeutic agent for refractory asthma according to the second aspect has less side effects since CXCL2 and CXCR2 are located at a downstream part of the signal transduction pathway and, among the immune cells, infiltration of neutrophils can be selectively inhibited.

### BRIEF DESCRIPTION OF THE DRAWINGS

FIG. 1 is a graph showing the results of the number of different types of immune cells in anti-CXCL2 antibody administration tests using a refractory asthma-induced model; and FIGS. 2(a) and 2(b) are views showing histopathological images of pulmonary tissues respectively in a group of mice administered with a control antibody and in a group of mice administered with an anti-CXCL2 antibody.

### PREFERRED MODE FOR CARRYING OUT THE INVENTION

Hereinafter, embodiments of the present invention will be described in detail. It will be understood that the embodiments are not intended to limit the present invention and may be implemented with any appropriate modifications within the scope of the object of the present invention.

<Method for Screening Prophylactic or Therapeutic Agents for Refractory Asthma>

The screening method according to the first aspect includes using, as an indicator, at least one selected from the group consisting of inhibition of CXCL2 or CXCR2 protein activity, inhibition of CXCL2 or CXCR2 gene expression, and inhibition of CXCL2 or CXCR2 protein expression, so that prophylactic or therapeutic agents for refractory asthma can be successfully screened. For prevention or treatment of refractory asthma, it is preferred to screen prophylactic or therapeutic agents for refractory asthma based on inhibition of neutrophilic infiltration in lung. The refractory asthma may include steroid-resistant refractory asthma. In view of the ability to selectively reduce neutrophils among immune cells, the refractory asthma is preferably neutrophil-dominant refractory asthma, more preferably, refractory asthma caused by neutrophil dominance. As used herein, the term “neutrophil-dominant” or “neutrophil dominance” means that neutrophils are dominant, specifically, the number of neutrophils is larger than that of any other type of immune cells. The screening method according to the first aspect also relates to a method for screening an inhibitor of neutrophilic infiltration in lung using the indicator defined above.

Examples of the CXCL2 or CXCR2 protein activity include migration, attraction, or infiltration of neutrophils to inflammatory sites, and cancer (e.g., liver cancer) metastasis enhancement (Otto Kollmar et al., Journal of Surgical Research, 145, 295-302 (2008)). The degree of the inhibition may be any statistically significant level. For example, the degree of the inhibition is preferably ¾ or less of, more preferably ⅔ or less of, even more preferably ½ or less of, the loss of the CXCL2 or CXCR2 protein activity, the CXCL2 or CXCR2 gene expression, or the CXCL2 or CXCR2 protein expression attainable in the absence of any test substance (e.g., in a system prior to administration of the test substance (e.g., a wild-type system) or in a negative control system (a control administered with a substance that does not affect the CXCL2 or CXCR2 protein activity, the CXCL2 or CXCR2 gene expression, or the CXCL2 or CXCR2 protein expression). The administration may be performed by methods known to those skilled in the art, such as intranasal administration, intratracheal administration, intraarterial injection, intravenous injection, and subcutaneous injection, among which intranasal administration or intratracheal administration is preferred, and intranasal administration is more preferred.

The screening method may be any type, such as an in vivo, in vitro, or in silico screening method, as long as it uses the indicator defined above. A preferred example of the screening method includes administering a refractory asthma-induced animal(s) with a test substance; and screening the test substance for a prophylactic or therapeutic agent for refractory asthma using, as an indicator, inhibition of CXCL2 or CXCR2 protein activity, inhibition of CXCL2 or CXCR2 gene expression, or inhibition of CXCL2 or CXCR2 protein expression in the animal(s) administered with the test substance compared with an asthma-induced animal(s) not administered with the test substance. Examples of the refractory asthma-induced animal include model mice with refractory asthma induced using complete Freund's adjuvant (CFA) (Bogaert et al., Am J Physiol Lung Cell Mol Physiol, 2011). Alternatively, the screening method may include culturing CXCL2 or CXCR2 gene-expressing cells in the presence and absence of a test substance; and screening the test substance for a prophylactic or therapeutic agent for refractory asthma using, as an indicator, inhibition of CXCL2 or CXCR2 protein activity, inhibition of CXCL2 or CXCR2 gene expression, or inhibition of CXCL2 or CXCR2 protein expression, in response to the presence and absence of the test substance.

The inhibition of CXCL2 or CXCR2 protein activity may be analyzed by measuring a reduction in the number of neutrophils in pulmonary tissues administered with the test substance relative to the number of neutrophils in pulmonary tissues not administered with the test substance. The level of mRNA expression for the CXCL2 or CXCR2 protein may be measured by a conventional method such as Northern blotting, Southern blotting, or RT-PCR. Specifically, the measurement can be performed using conventional methods known to those skilled in the art as described, for example, in Molecular Cloning 2nd Edition or Current Protocols in Molecular Biology. The level of expression of the CXCL2 or CXCR2 protein may also be measured by conventional immunoassay such as Western blotting or ELISA using an antibody. Specifically, the measurement may be performed using conventional methods known to those skilled in the art as described, for example, in Molecular Cloning 2nd Edition or Current Protocols in Molecular Biology.

Based on the CXCL2 or CXCR2 gene base sequence data, the expression of the CXCL2 or CXCR2 gene in various human tissues can also be detected even in silico. The expression of the CXCL2 gene in various human tissues can also be detected in vivo or in vitro, for example, using probes or primers having part or whole of the base sequence of the gene. The expression of the CXCL2 or CXCR2 gene may be detected by a conventional method such as RT-PCR, Northern blotting, or Southern blotting.

When PCR is performed, any primers capable of specifically amplifying only the CXCL2 or CXCR2 gene may be used, which may be appropriately selected based on the sequence data of the CXCL2 or CXCR2 gene. For example, the probes or primers may be an oligonucleotide and an antisense oligonucleotide, in which the oligonucleotide includes at least 10 consecutive nucleotides in the base sequence of the CXCL2 or CXCR2 gene or in the expression control region of the gene, and the antisense oligonucleotide has a sequence complementary to that of the oligonucleotide. More specifically, the oligonucleotide may have a base sequence of 10 to 60 consecutive residues, preferably 10 to 40 consecutive residues in the base sequence of the CXCL2 or CXCR2 gene or in the expression control region of the gene, and the antisense oligonucleotide may have a sequence complementary to that of the oligonucleotide.

The oligonucleotide and the antisense oligonucleotide may be produced by a conventional method using a DNA synthesizer. The oligonucleotide or the antisense oligonucleotide may be, for example, a sense primer corresponding to a partial 5′ terminal base sequence of mRNA to be detected or an antisense primer corresponding to a partial 3′ terminal base sequence of mRNA to be detected. The sense and antisense primers may be oligonucleotides having about 10 to about 60 bases, preferably about 10 to about 40 bases, with no extreme variations in melting temperature (Tm) or the number of bases. In the present invention, derivatives of the oligonucleotides may also be used, such as methylated or phosphorothioated oligonucleotides.

Any substance may be used as the test substance to be subjected to the screening method according to the first aspect of the present invention. The test substance may be any type, such as an antibody, a nucleic acid molecule, an individual low-molecular-weight synthetic compound, a compound occurring in a natural product extract, or a synthetic peptide. The test substance may also be an artificial nuclease for genome editing described later. Alternatively, the test compound may also be in a compound library, a phage display library, or a combinatorial library. Construction of compound libraries is known to those skilled in the art, and commercially available compound libraries may also be used. The test substance is preferably an antibody, a low-molecular-weight compound (e.g., a compound library), a nucleic acid molecule, or an artificial nuclease for genome editing. For high specificity for the CXCL2 or CXCR2 protein or the CXCL2 or CXCR2 gene, the test substance is more preferably an antibody, a low-molecular-weight compound, or a nucleic acid molecule, even more preferably an antibody or aptamer capable of selectively binding to the CXCL2 or CXCR2 protein, or a nucleic acid molecule having a sequence complementary to an oligonucleotide in the CXCL2 or CXCR2 gene (a coding region (CDS) or an untranslated region (UTR) of an exon or an intronic region) or in an expression control region of the gene, further more preferably an antibody or aptamer capable of selectively binding to the CXCL2 or CXCR2 protein.

CXCL2 Protein

The CXCL2 protein may be any one of the following proteins:
(a) A protein comprising an amino acid sequence set forth in SEQ ID NO: 1 or 2 of the Sequence Listing;
(b) A protein comprising an amino acid sequence having an amino acid deletion, substitution, and/or addition at one or several positions in the amino acid sequence set forth in SEQ ID NO: 1 or 2 of the Sequence Listing, the protein having activity to attract neutrophils to an inflammatory site, to cause neutrophils to infiltrate into an inflammatory site, or to promote cancer metastasis; and
(c) A protein comprising an amino acid sequence having at least 95% homology to the amino acid sequence set forth in SEQ ID NO: 1 or 2 of the Sequence Listing, the protein having activity to attract neutrophils to an inflammatory site, to cause neutrophils to infiltrate into an inflammatory site, or to promote cancer metastasis. The protein (a) is preferred in order to screen prophylactic or therapeutic agents for refractory asthma and to make it possible to directly use a human-derived protein with no need to undertake extra steps such as transformation. SEQ ID NO: 1 represents the amino acid sequence of a human CXCL2 protein. SEQ ID NO: 2 represents the amino acid sequence of a mouse CXCL2 protein.

Regarding the expression used herein “an amino acid sequence having an amino acid deletion, substitution, and/or addition at one or several positions in the amino acid sequence”, the range of “one to several” is preferably, but not limited to, 1 to about 10, more preferably 1 to about 5, even more preferably 1 to about 3. As used herein, the term “an amino acid sequence having at least 95% homology” means an amino acid sequence having a homology of 95% or more, preferably 96% or more, more preferably 97% or more. All proteins that are encoded by mutant genes highly homologous to the gene having the base sequence set forth in SEQ ID NO: 5 or 6 of the Sequence Listing and have activity to attract neutrophils to an inflammatory site, to cause neutrophils to infiltrate into an inflammatory site, or to promote cancer metastasis fall within the scope of the present invention. Although the side chains of amino acids that constitute the building blocks of proteins differ in hydrophobicity, charge, size, and so on, some highly conservative relationships in terms of having substantially no effect on the entire three-dimensional structure (also called steric structure) of a protein are known empirically and based on actual physicochemical measurements. For example, the amino acid residue substitution may be substitution between glycine (Gly) and proline (Pro), between Gly and alanine (Ala) or valine (Val), between leucine (Leu) and isoleucine (Ile), between glutamic acid (Glu) and glutamine (Gln), between aspartic acid (Asp) and asparagine (Asn), between cysteine (Cys) and threonine (Thr), between Thr and serine (Ser) or Ala, or between lysine (Lys) and arginine (Arg).

Therefore, all mutant proteins having an amino acid substitution, insertion, deletion, or the like at a position or positions in the CXCL2 amino acid sequence set forth in SEQ ID NO: 1 or 2 of the Sequence Listing will also fall within the scope of CXCL2 if the mutation is highly conservative in the three-dimensional structure of CXCL2 and the mutant proteins have activity to attract neutrophils to an inflammatory site, to cause neutrophils to infiltrate into an inflammatory site, or to promote cancer metastasis like CXCL2. The CXCL2 protein may be obtained using any method. The CXCL2 protein may be a chemically synthesized protein, a naturally-occurring protein isolated from a biological sample or cultured cells, or a recombinant protein produced using genetic recombination techniques.

CXCL2 Gene

The CXCL2 gene includes exon 1, intron 1, exon 2, intron 2, exon 3, intron 3, and exon 4, and this organization is highly conserved among humans, mice, and other mammals. SEQ ID NO: 3 represents a base sequence encoding complementary DNA (cDNA) for unspliced pre-mRNA for human CXCL2. SEQ ID NO: 4 represents a base sequence encoding cDNA for unspliced pre-mRNA for mouse CXCL2. Table 1 below shows a summary of the regions of exons 1 to 4 and introns 1 to 3 in each of cDNA for the pre-mRNA for the human CXCL2 represented by SEQ ID NO: 3 and cDNA for the pre-mRNA for the mouse CXCL2 represented by SEQ ID NO: 4.

TABLE 1

| | | Exon 1 | Intron 1 | Exon 2 | Intron 2 | Exon 3 | Intron 3 | Exon 4 |
|---|---|---|---|---|---|---|---|---|
| Human | SEQ ID | Position | Position | Position | Position | Position | Position | Position |
| | NO: 3 | 1 to 273 | 274 to 371 | 372 to 495 | 496 to 609 | 610 to 693 | 694 to 1522 | 1523 to 2259 |
| Mouse | SEQ ID | Position | Position | Position | Position | Position | Position | Position |
| | NO: 4 | 1 to 151 | 152 to 243 | 244 to 367 | 368 to 484 | 485 to 568 | 569 to 1316 | 1317 to 2066 |

Exons 1 to 4 also include coding regions (CDSs) that encode amino acids and untranslated regions (UTRs) that do not encode any amino acids. The UTRs in exons include 5′UTR upstream of the initiation codon and 3′UTR downstream of the termination codon. The human CXCL2 gene encoding the human CXCL2 mRNA has a sequence set forth in SEQ ID NO: 5 described below. In SEQ ID NO: 5, the base sequence from position 174 to position 497 is CDS, the base sequence from position 1 to position 173 is 5′UTR, and the base sequence from position 498 to position 1218 is 3′UTR.

The mouse CXCL2 gene encoding the mouse CXCL2 mRNA has a sequence set forth in SEQ ID NO: 6 described below. In SEQ ID NO: 6, the base sequence from position 73 to position 375 is CDS, the base sequence from position 1 to position 72 is 5′UTR, and the base sequence from position 376 to position 1109 is 3′UTR.

All genes encoding CXCL2 proteins (e.g., proteins having the amino acid sequence set forth in SEQ ID NO: 1 or 2) belong to the CXCL2 gene. Specifically, the CXCL2 gene may be gene (a) or (b) below. Gene (a) below is preferred in order to screen prophylactic or therapeutic agents for refractory asthma and to make it possible to directly use a human-derived gene with no need to undertake extra steps such as transformation.
(a) A gene comprising a base sequence set forth in SEQ ID NO: 5 or 6 of the Sequence Listing; and
(b) A gene comprising a base sequence having a base deletion, substitution, and/or addition at one or several positions in the base sequence set forth in SEQ ID NO: 5 or 6 of the Sequence Listing, the gene encoding a protein having activity to attract neutrophils to an inflammatory site, to cause neutrophils to infiltrate into an inflammatory site, or to promote cancer metastasis.

Regarding the expression used herein “a base sequence having a base deletion, substitution, and/or addition at one or several positions in the base sequence”, the range of “one or several” is preferably, but not limited to, 1 to about 20, more preferably 1 to about 10, even more preferably 1 to about 5. Regarding the degree of the DNA mutation, examples of mutant DNA include DNA having at least 80%, preferably at least 85%, more preferably at least 90%, even more preferably at least 95%, further more preferably at least 98% homology to the base sequence of the CXCL2 gene set forth in SEQ ID NO: 5 or 6 of the Sequence Listing.

CXCR2 Protein

The CXCR2 protein may be any of the following proteins:
(a) A protein comprising an amino acid sequence set forth in SEQ ID NO: 7 or 8 of the Sequence Listing;
(b) A protein comprising an amino acid sequence having an amino acid deletion, substitution, and/or addition at one or several positions in the amino acid sequence set forth in SEQ ID NO: 7 or 8 of the Sequence Listing, the protein having activity to promote migration of neutrophils to an inflammatory site; and
(c) A protein comprising an amino acid sequence having at least 95% homology to the amino acid sequence set forth in SEQ ID NO: 7 or 8 of the Sequence Listing, the protein having activity to promote migration of neutrophils to an inflammatory site. The protein (a) is preferred in order to screen prophylactic or therapeutic agents for refractory asthma and to make it possible to directly use a human-derived protein with no need to undertake extra steps such as transformation. SEQ ID NO: 7 represents the amino acid sequence of a human CXCR2 protein. SEQ ID NO: 8 represents the amino acid sequence of a mouse CXCR2 protein.

All proteins that are encoded by mutant genes highly homologous to the gene having the base sequence set forth in SEQ ID NO: 11 or 12 of the Sequence Listing and have activity to promote migration of neutrophils to an inflammatory site fall within the scope of the present invention.

All mutant proteins having an amino acid substitution, insertion, deletion, or the like at a position or positions in the CXCR2 amino acid sequence set forth in SEQ ID NO: 7 or 8 of the Sequence Listing will also fall within the scope of CXCR2 if the mutation is highly conservative in the three-dimensional structure of CXCR2 and the mutant proteins have activity to attract neutrophils to an inflammatory site, to cause neutrophils to infiltrate into an inflammatory site, or to promote cancer metastasis like CXCR2. The CXCR2 protein may be obtained using any method. The CXCR2 protein may be a chemically synthesized protein, a naturally-occurring protein isolated from a biological sample or cultured cells, or a recombinant protein produced using genetic recombination techniques.

CXCR2 Gene

The human CXCR2 gene includes exon 1, intron 1, exon 2, intron 2, and exon 3, and the mouse CXCR2 gene includes exon 1, intron 1, and exon 2. SEQ ID NO: 9 represents a base sequence encoding complementary DNA (cDNA) for unspliced pre-mRNA for human CXCR2. SEQ ID NO: 10 represents a base sequence encoding cDNA for unspliced pre-mRNA for mouse CXCR2. Table 2 below shows a summary of the regions of exons 1 to 3 and introns 1 and 2 in each of cDNA for the pre-mRNA for the human CXCR2 represented by SEQ ID NO: 9 and cDNA for the pre-mRNA for the mouse CXCR2 represented by SEQ ID NO: 10.

TABLE 2

| | | Exon 1 | Intron 1 | Exon 2 | Intron 2 | Exon 3 |
|---|---|---|---|---|---|---|
| Human | SEQ ID | Position | Position | Position | Position | Position |
| | NO: 9 | 1 to 350 | 351 to 3310 | 3311 to 3362 | 3363 to 8773 | 8774 to 11250 |
| Mouse | SEQ ID | Position | Position | Position | Absent | Absent |
| | NO: 10 | 1 to 129 | 130 to 4339 | 4340 to 7256 | | |

The human CXCR2 gene encoding the human CXCR2 mRNA has a sequence set forth in SEQ ID NO: 11 described below. The mouse CXCR2 gene encoding the mouse CXCR2 mRNA has a sequence set forth in SEQ ID NO: 12 described below.

All genes encoding CXCR2 proteins (e.g., proteins having the amino acid sequence set forth in SEQ ID NO: 7 or 8) belong to the CXCR2 gene. Specifically, the CXCR2 gene may be gene (a) or (b) below. Gene (a) below is preferred in order to screen prophylactic or therapeutic agents for refractory asthma and to make it possible to directly use a human-derived gene with no need to undertake extra steps such as transformation.
(a) A gene comprising a base sequence set forth in SEQ ID NO: 11 or 12 of the Sequence Listing; and
(b) A gene comprising a base sequence having a base deletion, substitution, and/or addition at one or several positions in the base sequence set forth in SEQ ID NO: 11 or 12 of the Sequence Listing, the gene encoding a protein having activity to promote migration of neutrophils to an inflammatory site.

How to Obtain CXCL2 or CXCR2 Gene

The CXCL2 or CXCR2 gene may be obtained by any process. The CXCL2 or CXCR2 gene may be isolated by a process including: preparing appropriate probes or primers based on the amino acid and base sequence data set forth in SEQ ID NOS: 1 to 12 of the Sequence Listing herein; and selecting desired clones from a human cDNA library (prepared in a conventional manner from appropriate cells in which the CXCL2 or CXCR2 gene is expressed). The CXCL2 or CXCR2 gene may also be obtained using a PCR technique. For example, PCR may be performed using chromosomal DNA or cDNA library derived from cultured human cells for templates and using a pair of primers designed to allow amplification of the base sequence set forth in SEQ ID NO: 5 or 6 or SEQ ID NO: 11 or 12. The PCR conditions may be selected as appropriate, which include, for example, 30 cycles of a reaction process of 94° C. for 30 seconds (denaturation), 55° C. for 30 seconds to 1 minute (annealing), and 72° C. for 2 minutes (extension), followed by reaction at 72° C. for 7 minutes. Subsequently, the amplified DNA fragments may be cloned into a suitable vector that can be amplified in a host such as E. coli. Preparation of the probes or primers, construction of cDNA libraries, screening of cDNA libraries, cloning of genes of interest, and other procedures are known to those skilled in the art and can be performed, for example, according to the methods described in Molecular Cloning 2nd Edition or Current Protocols in Molecular Biology.

A gene (mutant gene) encoding a protein that comprises a base sequence having a base deletion, substitution, and/or addition at one or several positions in the base sequence set forth in SEQ ID NO: 5 or 6 or SEQ ID NO: 11 or 12 of the Sequence Listing herein and has activity to attract neutrophils to an inflammatory site, to cause neutrophils to infiltrate into an inflammatory site, or to promote cancer metastasis as mentioned above may also be produced using chemical synthesis, genetic engineering techniques, mutagenesis, or any other methods known to those skilled in the art. For example, mutant DNA may be obtained by introducing mutations into DNA having the base sequence set forth in SEQ ID NO: 5 or 6 or SEQ ID NO: 11 or 12. Specifically, mutations may be introduced into DNA having the base sequence set forth in SEQ ID NO: 5 or 6 or SEQ ID NO: 11 or 12 using a method of bringing a mutagenic drug into contact with the DNA to allow it to act on the DNA, ultraviolet irradiation techniques, genetic engineering techniques, or other methods. Site-directed mutagenesis is a genetic engineering technique, which is useful because of its ability to introduce a specific mutation into a specific site. Site-directed mutagenesis can be performed, for example, according to the methods described in Molecular Cloning 2nd Edition or Current Protocols in Molecular Biology.

The DNA having the CXCL2 or CXCR2 gene base sequence set forth in SEQ ID NO: 5 or 6 or SEQ ID NO: 11 or 12 of the Sequence Listing may be partially modified by various artificial processes such as site-directed mutagenesis, random mutagenesis by mutagen treatment, and mutation, deletion, and ligation of DNA fragments through restriction enzyme fragmentation. If the resulting DNA mutants encode a protein having activity to attract neutrophils to an inflammatory site, to cause neutrophils to infiltrate into an inflammatory site, or to promote cancer metastasis, they will also fall within the scope of the CXCL2 or CXCR2 gene no matter how they differ from the DNA sequence set forth in SEQ ID NO: 5 or 6 or SEQ ID NO: 11 or 12.

<Prophylactic or Therapeutic Agent for Refractory Asthma>

The prophylactic or therapeutic agent for refractory asthma according to the second aspect (hereinafter also simply referred to as “the prophylactic or therapeutic agent according to the second aspect”) includes an inhibitor of CXCL2 or CXCR2 protein activity, an inhibitor of CXCL2 or CXCR2 gene expression, or an inhibitor of CXCL2 or CXCR2 protein expression. The prevention or treatment of refractory asthma is preferably based on inhibition of neutrophilic infiltration in lung. The refractory asthma may include steroid-resistant refractory asthma. In view of the ability to selectively reduce neutrophils among immune cells, the refractory asthma is preferably neutrophil-dominant refractory asthma, more preferably, refractory asthma caused by neutrophil dominance. The prophylactic or therapeutic agent according to the second aspect also relates to an inhibitor of neutrophilic infiltration in lung.

Antibody or Aptamer Capable of Selectively Binding to CXCL2 or CXCR2 Protein

The inhibitor of CXCL2 or CXCR2 protein activity may be an antibody, a macromolecular compound (such as a nucleic acid), a low-molecular-weight compound, or any other substance that inhibits the activity of the CXCL2 or CXCR2 protein.

A preferred example of the inhibitor of CXCL2 or CXCR2 protein activity includes a prophylactic or therapeutic agent for refractory asthma including an antibody that selectively binds to the CXCL2 or CXCR2 protein. The antibody may be any polyclonal or monoclonal antibody that can specifically bind to the CXCL2 or CXCR2 protein. Polyclonal antibodies can be prepared by separating and purifying serum obtained from an animal immunized with an antigen. Monoclonal antibodies can be prepared by a process including: fusing myeloma cells and antibody-producing cells obtained from an animal immunized with an antigen to produce hybridomas; culturing the hybridomas or administering the hybridomas to an animal to induce ascites tumor in the animal; and separating and purifying the culture medium or ascites fluid. Antigens can be prepared by a process including: purifying the CXCL2 or CXCR2 protein from various cultured human cells; or introducing, into a host such as E. coli, yeasts, animal cells, or insect cells, a recombinant vector containing DNA encoding a protein having the amino acid sequence of the CXCL2 or CXCR2 protein, a mutation of the sequence, or a part thereof; and separating and purifying a protein resulting from the expression of the DNA. Antigens can also be prepared by synthesizing peptides having a part of the amino acid sequence of the CXCL2 or CXCR2 protein using an amino acid synthesizer. Immunization methods may include administering antigens directly to non-human mammals such as rabbits, goats, rats, mice, or hamsters subcutaneously, intravenously, or intraperitoneally. Preferably, immunization methods include administering antigens coupled to a highly antigenic carrier protein such as Fissurellidae hemocyanin, keyhole limpet hemocyanin, bovine serum albumin, or bovine thyroglobulin; or administering antigens together with a suitable adjuvant such as complete Freund's adjuvant, aluminum hydroxide gel, or pertussis vaccine.

Antigens may be administered once and then administered 3 to 10 times every 1 to 2 weeks. Three to seven days after each administration, blood may be collected from the venous plexus of the fundus, and then measured for antibody titer according to enzyme immunoassay or other methods to determine whether or not the serum reacts with the antigen used for immunization. The non-human mammal of which the serum has a sufficient antibody titer to the antigen used for immunization may be used as a source of serum or antibody-producing cells. Polyclonal antibodies may be prepared by separating and purifying the serum mentioned above. Monoclonal antibodies may be prepared by a process including: fusing the antibody-producing cells and myeloma cells derived from a non-human mammal to produce hybridomas; culturing the hybridomas or administering the hybridomas to an animal to induce ascites tumor in the animal; and separating and purifying the culture medium or ascites fluid. The antibody-producing cells may be splenic cells, lymph node cells, or antibody-producing cells in peripheral blood, and preferably splenic cells.

The myeloma cells may be established mouse cell lines such as 8-azaguanine resistant mouse (BALB/c derived) myeloma cell lines including P3-X63Ag8-U1 (P3-U1 (Current Topics in

Microbiology and Immunology, 18, 1-7 (1978)), P3-NS1/1-Ag41 (NS-1) (European J. Immunology, 6, 511-519 (1976)), SP2/0-Ag14 (SP-2) (Nature, 276, 269-270 (1978)), P3-X63-Ag8653 (653) (J. Immunology, 123, 1548-1550 (1979)), and P3-X63-Ag8 (X63) (Nature, 256, 495-497 (1975)). Hybridoma cells can be prepared by the following process. First, antibody-producing cells and myeloma cells are mixed and suspended in a HAT medium (a medium prepared by adding hypoxanthine, thymidine, and aminopterin to a normal medium), and then cultured for 7 to 14 days. After the culture, part of the culture supernatant is sampled and subjected to enzyme immunoassay or other measurement methods so that samples reactive to the antigen and not reactive to proteins not including the antigen are selected. Cloning is then performed using a limiting dilution method, and cells that are found to have a high and stable antibody titer as measured by enzyme immunoassay are selected as monoclonal antibody-producing hybridoma cells. Monoclonal antibodies can be prepared by separating and purifying a culture medium obtained by culturing hybridoma cells, or by separating and purifying an ascites fluid obtained from the peritoneal cavity of an animal intraperitoneally administered with hybridoma cells to induce ascites tumor in the animal.

Methods for separating and purifying polyclonal or monoclonal antibodies may include one or any combination of centrifugation, ammonium sulfate precipitation, caprylic acid precipitation, and chromatography using a DEAE-Sepharose column, an anion exchange column, a protein A or G column, or a gel filtration column. As used herein, the term “antibody” is intended to include not only a full-length antibody but also any fragment of the antibody. Antibody fragments are preferably functional fragments, such as F(ab′)₂ and Fab′. F (ab′)₂ and Fab′ are produced by treating an immunoglobulin with a protease (e.g., pepsin or papain), which are antibody fragments produced by digestion at sites upstream and downstream of the disulfide bond between two H chains in the hinge region.

The antibody to be administered to humans is preferably a humanized or human-like antibody in order to have reduced immunogenicity. Such a humanized or human-like antibody may be produced using a mammal such as a transgenic mouse. Humanized antibodies are described, for example, in Morrison, S. L. et al., Proc. Natl. Acad. Sci. USA, 81:6851-6855 (1984); and Hiroshi Noguchi, Igaku no Ayumi (in Japanese) 167:457-462 (1993). Humanized chimeric antibodies may be produced using gene recombination to link the V region of a mouse antibody with the C region of a human antibody. Humanized antibodies may be produced by substituting a human antibody-derived sequence for a region of a mouse monoclonal antibody other than its complementarity determining region (CDR). The antibody may also be used in the form of an immobilized antibody, which has the antibody immobilized on an insoluble carrier such as a solid phase carrier, or in the form of a labeled antibody, which has the antibody labeled with a labeling substance. All of such immobilized antibodies and labeled antibodies also fall within the scope of the present invention.

Among the antibodies described above, the antibody capable of specifically binding to the CXCL2 or CXCR2 protein to inhibit its activity may be used as a prophylactic or therapeutic agent for refractory asthma.

The antibody may be used to form a pharmaceutical composition as a prophylactic or therapeutic agent for refractory asthma. In this case, the pharmaceutical composition may be prepared using the antibody as an active ingredient and using a pharmaceutically acceptable carrier, diluent (e.g., an immunogenic adjuvant), stabilizer, or excipient, or other materials. The prophylactic or therapeutic agent for refractory asthma including the antibody may be formulated into a dosage form by a process including sterilization by filtration, lyophilization, and transfer to a dose vial or stabilization into an aqueous preparation.

Another preferred example of the inhibitor of the CXCL2 or CXCR2 protein may be a prophylactic or therapeutic agent for refractory asthma including an aptamer that selectively binds to the CXCL2 or CXCR2 protein. The term “aptamer” refers to a nucleic acid drug including single-stranded RNA or DNA, which has a three-dimensional structure to enable it to bind to a target protein to inhibit the function of the protein. Aptamers have a high ability to bind to the target protein, high specificity, low immunogenicity, and high storage stability, and can be produced by chemical synthesis. The base length of the aptamer that selectively binds to the CXCL2 or CXCR2 protein is not limited as long as it specifically binds to the CXCL2 or CXCR2 protein. Preferably, the aptamer has 15 to 60 bases, more preferably 20 to 50 bases, even more preferably 25 to 47 bases, further more preferably 26 to 45 bases. The SELEX (systematic evolution of ligands by exponential enrichment) method may be used to obtain the aptamer that selectively binds to the CXCL2 or CXCR2 protein.

The prophylactic or therapeutic agent for refractory asthma according to the second aspect may be administered to patients by methods known to those skilled in the art, such as intranasal administration, intratracheal administration, intraarterial injection, intravenous injection, and subcutaneous injection, and preferably administered by intranasal administration or intratracheal administration, more preferably intranasal administration. A person skilled in the art can appropriately select a suitable dosage although the dosage varies depending on the body weight and age of the patient and the administration method. The dosage of the antibody or aptamer as an active ingredient is generally in the range of about 0.1 μg to about 100 mg per kg body weight per dose.

Antisense Oligonucleotide

The inhibitor of CXCL2 or CXCR2 gene expression or the inhibitor of CXCL2 or CXCR2 protein expression may be an antisense oligonucleotide as described above, which has a sequence complementary to an oligonucleotide in the CXCL2 or CXCR2 gene (CDS or UTR in an exon or an intron) or in the expression control region of the CXCL2 or CXCR2 gene. The antisense oligonucleotide may be introduced into cells to inhibit the transcription or translation of the CXCL2 or CXCR2 gene so that refractory asthma can be prevented or treated. For example, after being introduced into cells, the antisense oligonucleotide, which is complementary to an oligonucleotide in the CXCL2 or CXCR2 gene (CDS or UTR in an exon or an intron) or in the expression control region of the CXCL2 or CXCR2 gene, hybridizes with the oligonucleotide to form a hybrid double strand. A nuclease (e.g., RNase H) specific for the hybrid double strand can degrade CXCL2 or CXCR2 mRNA to inhibit the transcription or translation of the CXCL2 or CXCR2 gene. The antisense oligonucleotide preferably has a sequence complementary to an oligonucleotide including at least 10 consecutive nucleotides, more preferably at least 11 consecutive nucleotides, even more preferably at least 12 consecutive nucleotides, further more preferably at least 13 consecutive nucleotides, still more preferably at least 14 consecutive nucleotides in the base sequence of the CXCL2 or CXCR2 gene (CDS or UTR in an exon or an intron) or in the expression control region of the CXCL2 or CXCR2 gene. Regarding the upper limit of the base length of the antisense oligonucleotide, the antisense oligonucleotide preferably has a sequence complementary to an oligonucleotide including at most 40 consecutive nucleotides, more preferably at most 30 consecutive nucleotides, even more preferably at most 25 consecutive nucleotides, further more preferably at most 20 consecutive nucleotides, still more preferably at most 17 consecutive nucleotides in the base sequence of the CXCL2 or CXCR2 gene (CDS or UTR in an exon or an intron) or in the expression control region of the CXCL2 or CXCR2 gene.

The antisense oligonucleotide preferably includes at least one nucleotide having at least one structure selected from the group consisting of a phosphorothioate structure, a cross-linked structure, and an alkoxy structure. For example, the antisense oligonucleotide may have a phosphorothioate structure in the phosphodiester bond moiety between nucleotides so that it can have nuclease resistance and increased hydrophobicity, which results in higher uptake into cells or nuclei. The antisense oligonucleotide may also have, in the nucleotide sugar moiety, a cross-linked structure such as 2′,4′-BNA (2′,4′-bridged nucleic acid, also called LNA (locked nucleic acid)), ENA (2′-O, 4′-C-ethylene-bridged nucleic acid); or an alkoxy structure such as a 2′-O-methylated structure or a 2′-O-methoxyethylated (2′-MOE) structure, so that it can have nuclease resistance and a higher ability to bind to mRNA. The antisense oligonucleotide preferably has a phosphorothioate structure in at least one phosphodiester bond moiety between nucleotides, more preferably phosphorothioate structures in at least 50% of the phosphodiester bond moieties, even more preferably phosphorothioate structures in at least 70% of the phosphodiester bond moieties, further more preferably phosphorothioate structures in at least 90% of the phosphodiester bond moieties, most preferably phosphorothioate structures in all of the phosphodiester bond moieties. The antisense oligonucleotide preferably has a cross-linked structure or an alkoxy structure at least at one end, more preferably cross-linked structures or alkoxy structures at both ends (what is called a gapmer antisense oligonucleotide), even more preferably cross-linked structures or alkoxy structures in two terminal regions each independently from the end to the fourth base from the end, further more preferably cross-linked structures or alkoxy structures at two or three bases from the end.

According to one embodiment, a method for introducing the antisense oligonucleotide into cells includes inserting the antisense oligonucleotide into a suitable vector and introducing the vector into suitable host cells. The suitable vector may be any type, such as a self-replicating vector (e.g., plasmid). Preferably, the vector is such that, when introduced into a host cell, it can be incorporated into the genome of the host cell and replicated together with the chromosome in which it has been incorporated. Examples of the vector suitable for use include E. coli-derived plasmids (e.g., pBR322, pUC118, etc.), B. subtilis-derived plasmids (e.g., pUB110, pSH19, etc.), and animal viruses such as bacteriophages, retroviruses, and vaccinia viruses. For recombination, a suitable synthetic DNA adapter may be used to add a translation initiation codon or a translation termination codon.

If necessary, the antisense oligonucleotide may be functionally linked to a suitable terminator such as a human growth hormone terminator or a TPI1 or ADH3 terminator for a fungal host. The recombinant vector may further include a polyadenylation signal (e.g., one derived from SV40 or adenoviral 5E1b region), a transcription enhancer sequence (e.g., SV40 enhancer), a translational enhancer sequence (e.g., one encoding adenoviral VARNA), and any other element. The recombinant vector may further include a DNA sequence that allows the vector to replicate in the host cell. An example of such a vector is the SV40 replication origin (for mammalian host cells). The recombinant vector may further include a selective marker. The selective marker may be, for example, a gene of which a complement is missing in the host cell, such as a dihydrofolate reductase (DHFR) or Schizosaccharomyces pombe TPI gene, or a drug-resistant gene such as a gene resistant to ampicillin, kanamycin, tetracycline, chloramphenicol, neomycin, or hygromycin.

Examples of the host cell into which the antisense oligonucleotide or the vector containing the same is introduced include higher eukaryotic cells, bacteria, yeasts, fungi, etc., among which mammalian cells are preferred. Examples of mammalian cells include HEK293 cells, HeLa cells, COS cells (e.g., COS-7 cells), BHK cells, CHL or CHO cells, and BALB/c mouse cells (e.g., BALB/c mouse fetal fibroblasts). Methods for transforming a mammalian cell by introduction of a gene into the cell to express the introduced gene are also known, such as lipofection methods, electroporation methods, and calcium phosphate methods.

The prophylactic or therapeutic agent according to the second aspect may or may not further include a carrier for lipofection for improving uptake into cells. Examples of the carrier for lipofection include carriers having high affinity for cell membranes (e.g., liposomes, cholesterol), among which Lipofectamine or Lipofectin is preferred, and Lipofectamine is more preferred. For example, the antisense oligonucleotide may be administered together with the carrier for lipofection to an affected area of the patient or systemically to the patient by injection, so that the antisense oligonucleotide can be incorporated into patient's cells to inhibit the expression of the CXCL2 or CXCR2 gene, thereby preventing or treating refractory asthma. When the antisense oligonucleotide has at least one structure selected from the group consisting of a phosphorothioate structure, a cross-linked structure, and an alkoxy structure, and is used in combination with the carrier for lipofection, the antisense oligonucleotide can be incorporated in an improved manner into patient's cells or nuclei. The dosage of the antisense oligonucleotide as an active ingredient is generally in the range of about 0.1 μg to about 100 mg per kg body weight per dose.

siRNA

The inhibitor of CXCL2 or CXCR2 gene expression or the inhibitor of CXCL2 or CXCR2 protein expression may also be a double-stranded RNA (siRNA (small interfering RNA)) including at least 20 consecutive nucleotides of CDS or UTR in the base sequence of RNA transcribed from the base sequence of the CXCL2 or CXCR2 gene, or may also be a DNA encoding the double-stranded RNA. The inhibitor is preferably a double-stranded RNA including at least 21 consecutive nucleotides of CDS or UTR in the base sequence of RNA transcribed from the base sequence of the CXCL2 or CXCR2 gene, or preferably a DNA encoding the double-stranded RNA. The inhibitor is preferably a double-stranded RNA including at most 30 consecutive nucleotides of CDS or UTR in the base sequence of RNA transcribed from the base sequence of the CXCL2 or CXCR2 gene, or preferably a DNA encoding the double-stranded RNA, and is more preferably a double-stranded RNA including at most 25 consecutive nucleotides of CDS or UTR in the base sequence of RNA transcribed from the base sequence of the CXCL2 or CXCR2 gene, or more preferably a DNA encoding the double-stranded RNA.

RNAi (RNA interference) refers to an event in which, when a double-stranded RNA (dsRNA) including a portion of mRNA encoding a portion of a certain target gene is introduced into a cell, the expression of the target gene is inhibited. The DNA encoding the double-stranded RNA may be, for example, a DNA having an inverted repeat of the sequence or a partial sequence of the CXCL2 or CXCR2 gene. The DNA having such an inverted repeat sequence may be introduced into mammalian cells to express the inverted repeat sequence of the target gene in the cells, so that the RNAi effect can be produced to inhibit the expression of the target gene (CXCL2 or CXCR2). The term “inverted repeat sequence” refers to a sequence including the sequence of a target gene and an inverted sequence of the target gene, in which the sequence and the inverted sequence are arranged in parallel with a suitable sequence in between them. Specifically, assume that the target gene is a double strand having a sequence of n bases as shown below.
5′−X₁X₂. . . Xₙ₋₁Xₙ−3′
3′−Y₁Y₂. . . Yₙ₋₁Yⁿ−5′
   The inverted sequence has the following sequence:
5′-YₙYₙ₋₁. . . Y₂Y₁−3′
3′−XₙXₙ₋₁. . . X₂X₁−5′
   wherein the bases represented by X and Y with the same numerical subscript are complementary.

The inverted repeat sequence includes the two types of sequences arranged with a suitable sequence in between them. For the inverted repeat sequence, two cases may occur: a case in which the sequence of the target gene is upstream of the inverted sequence; and a case in which the inverted sequence is upstream of the sequence of the target gene. The inverted repeat sequence used in the present invention may be either one of the two types. Preferably, the inverted sequence is upstream of the sequence of the target gene. The sequence in between the sequence of the target gene and the inverted sequence may be a region that forms a hairpin loop (small hairpin RNA (shRNA)) when transcribed into RNA. This region may have any length that allows the formation of a hairpin loop. Preferably, the length of this region is 0 to about 300 bp, more preferably 0 to about 100 bp. The sequence of this region may have a restriction enzyme site.

In the present invention, the inverted repeat sequence of the target gene may be incorporated downstream of a promoter sequence operable in a mammal so that the inverted repeat sequence of the target gene can be expressed in the mammal cell. In the invention, the promoter may have any sequence that makes it operable in a mammal.

For example, the double-stranded RNA or DNA may be administered together with the carrier for lipofection, which is used to assist the uptake into cells, to an affected area of the patient or systemically to the patient by injection or the like, so that the RNA or DNA can be incorporated into patient's cells to control severe asthma. The dosage of the double-stranded RNA or DNA as an active ingredient is generally in the range of about 0.1 μg to about 10 mg per kg body weight per dose.

Artificial Nuclease

The inhibitor of CXCL2 gene or protein expression may also be an artificial nuclease for genome editing, such as the CRISPR (clustered regularly interspaced short palindromic repeats)/Cas nuclease, or an artificial restriction enzyme (artificial nuclease) produced using transcription activator-like effector nuclease (TALEN) and zinc finger nuclease (ZFN). TALEN is an artificial nuclease including a DNA cleavage domain and TALEs which are domains formed by polymerization of four types of units that respectively recognize and bind to four bases (A, T, G, and C), in which TALEs can recognize and bind to at least a partial sequence in the CXCL2 or CXCR2 gene. ZFN is an artificial nuclease in the form of a chimeric protein including a zinc finger domain and a DNA cleavage domain. The zinc finger domain has a structure including linked zinc finger units that recognize specific three-base sequences, which can recognize and bind to DNA sequences of multiples of three bases. The zinc finger domain can recognize and bind to at least a partial sequence in the CXCL2 or CXCR2 gene.

The CRISPR/Cas nuclease includes a guide RNA and a Cas nuclease (preferably Cas9). The term “guide RNA” means RNA that has the function of binding to a Cas nuclease, a DNA cleavage enzyme, to guide the Cas nuclease to a target DNA (at least a partial sequence in the CXCL2 or CXCR2 gene). The guide RNA has a 5′-end sequence complementary to the target DNA (at least a partial sequence in the CXCL2 or CXCR2 gene). The guide RNA binds to the target DNA through the complementary sequence to guide a Cas nuclease to the target DNA. The Cas nuclease functions as a DNA endonuclease to cleave DNA at a site where the target DNA exists, and thus can specifically reduce, for example, the expression of the CXCL2 or CXCR2 gene. The at least partial sequence in the target CXCL2 or CXCR2 gene is preferably 15 to 25 bases long, more preferably 17 to 22 bases long, even more preferably 18 to 21 bases long, further more preferably 20 bases long.

Eukaryotic cells or eukaryotes containing the CXCL2 or CXCR2 gene may be transfected with a guide RNA specific for the CXCL2 or CXCR2 gene or with a DNA encoding the guide RNA and transfected with a nucleic acid encoding a Cas nuclease or a composition containing a Cas nuclease, so that the expression of the CXCL2 or CXCR2 gene can be reduced. A Cas nuclease or a nucleic acid encoding a Cas nuclease and a guide RNA or a DNA encoding a guide RNA may be delivered into cells using various methods known in the art. Examples of such methods include, but are not limited to, microinjection, electroporation, DEAE-dextran treatment, lipofection, nanoparticle-mediated transfection, protein transduction domain-mediated transduction, virus-mediated gene delivery, and PEG-mediated transfection into protoplasts. A Cas nuclease or a nucleic acid encoding a Cas nuclease and a guide RNA may be delivered into an organism by injection or various other methods known in the art for administration of genes or proteins. A Cas protein or a nucleic acid encoding a Cas nuclease and a guide RNA may be delivered separately or in the form of a complex of the nucleic acid or protein and the guide RNA into cells. A Cas nuclease fused to a protein transduction domain such as Tat can also be efficiently delivered into cells. Preferably, eukaryotic cells or eukaryotes are co-transfected or sequentially transfected with Cas9 nuclease and a guide RNA. Sequential transfection may include first transfection with a nucleic acid encoding a Cas nuclease and then second transfection with a naked guide RNA. The second transfection is, preferably, but not limited to, transfection after 3, 6, 12, 18, or 24 hours. A guide RNA expression unit may also be used to express the guide RNA. The guide RNA expression unit is preferably a CRISPR-Cas9-based transcription unit including the target sequence (a partial sequence of the CXCL2 or CXCR2 gene) and a guide RNA, preferably a unit including a promoter region for expression of a guide RNA (an RNA polymerase III promoter (e.g., a promoter selected from U6 and H1 promoters)), the target sequence (the CXCL2 or CXCR2 gene), and a guide RNA, more preferably a unit including a promoter, a sequence complementary to the target sequence (at least a partial sequence of the CXCL2 or CXCR2 gene), and a guide RNA, which are seamlessly linked together. In order to prevent off-targeting, the CRISPR/Cas nuclease may include a Cas9 variant that acts as a nickase to cut only one strand of a double-stranded DNA. The single-strand cutting Cas9 variant is, for example, Cas9 (D10A). For example, when a guide RNA having a target sequence complementary to one strand of the target DNA is used in combination with a guide RNA having a target sequence complementary to another strand very close to the one strand, the single-strand cutting Cas9 variant can cut the one strand with 20 base specificity and the other strand with 20 base specificity and thus can cut the DNA with 40 base specificity in total, which can considerably increase the target specificity.

The dosage of the artificial nuclease as an active ingredient or the nucleic acid as an active ingredient encoding the artificial nuclease is generally in the range of about 0.1 μg to about 10 mg per kg body weight per dose.

The prophylactic or therapeutic agent for refractory asthma according to the second aspect may be administered orally or parenterally and administered systemically or topically. Examples of parenteral administration include intranasal administration, intratracheal administration, intravenous injection such as intravenous infusion, intramuscular injection, intraperitoneal injection, and subcutaneous injection, among which intranasal administration or intratracheal administration is preferred, intranasal administration is more preferred. The administration method may be selected as appropriate according to the age and symptoms of the patient. While the dosage varies depending on the age, the route of administration, and the number of times of administration, a person skilled in the art can select appropriate dosages. Dosage forms suitable for parenteral administration include, for example, formulations containing a stabilizer, a buffer, a preservative, an isotonizing agent, or other additives, which may further contain a pharmaceutically acceptable carrier or additive. Examples of such a carrier or additive include, but are not limited to, water, organic solvents, polymeric compounds (such as collagen, and polyvinyl alcohol), stearic acid, human serum albumin (HSA), mannitol, turbitol, lactose, and surfactants.

### EXAMPLES

Hereinafter, the present invention will be described more specifically with reference to Examples, which are not intended to limit the scope of the present invention.

<<Anti-CXCL2 Antibody Administration Test Using Refractory Asthma Model>>

On day 0, female 7- to 8-week-old, wild-type BALB/c mice were subcutaneously sensitized with 50 μl of complete Freund's adjuvant (CFA from Sigma-Aldrich) and 20 μg of ovalbumin (OVA from Sigma-Aldrich) emulsified in 50 μl of PBS. On day 21, the mice were slightly anesthetized and then administered with a total volume of 100 μl of nasal drops including 50 μg of a rat anti-mouse CXCL2 antibody (manufactured by R & D) or 50 μg of an isotype control antibody (rat IgG2b manufactured by R & D) (8 mice/group). After 1 hour, all mice (8 mice/group) were allowed to inhale an aerosol including 3% OVA in PBS for 20 minutes. In a control group, the mice were allowed to inhale a PBS aerosol for 20 minutes (1 mouse/group). Six hours after the inhalation, bronchoalveolar lavage fluid and lung samples were collected from the mice. The obtained lung samples were used for the histopathological analysis of lung tissue described below.

<Counting of Various Types of Immune Cells in Bronchoalveolar Lavage Fluid>

Various types of immune cells (macrophages, lymphocytes, neutrophils, and eosinophils) were counted in the above-mentioned bronchoalveolar lavage fluid. The results are shown in

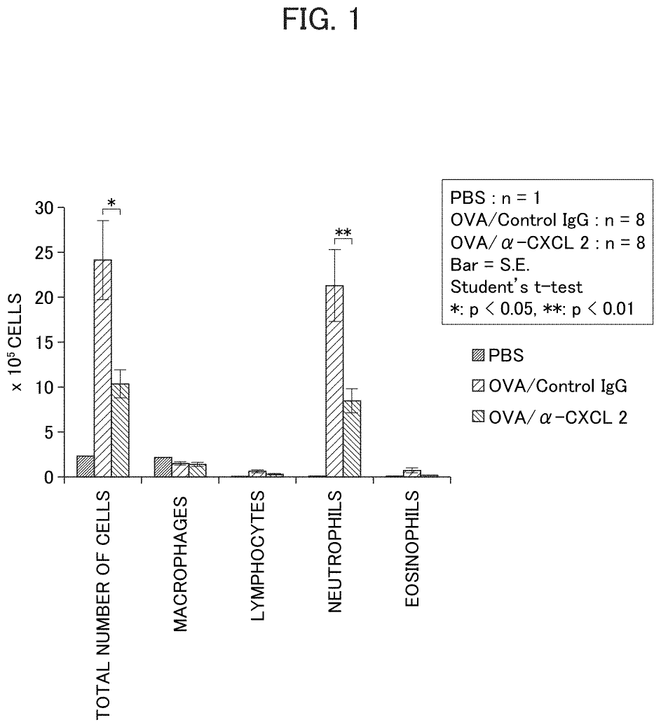

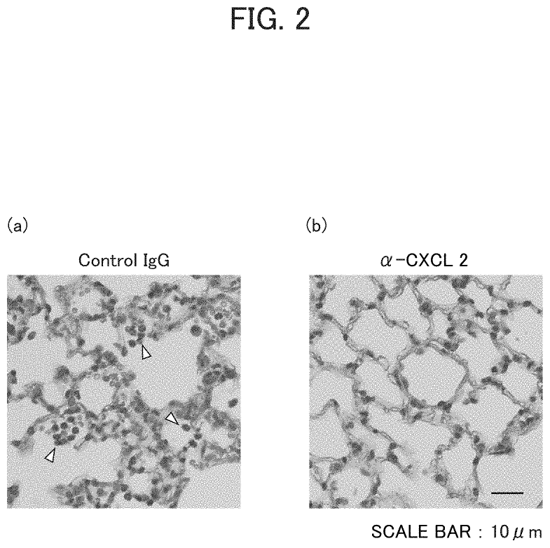

FIG. 1. In FIG. 1, the PBS group shows the number of cells in mice exposed to PBS aerosol as a control, the OVA/control IgG group shows the number of cells in mice exposed to OVA aerosol after the administration of the control antibody in advance, and the OVA/α-CXCL2 group shows the number of cells in mice exposed to OVA aerosol after the administration of the anti-CXCL2 antibody in advance. As is evident from the results shown in FIG. 1, the number of neutrophils in the refractory asthma model group was increased compared to the control group, which indicates OVA sensitization is appropriately done. The number of neutrophils of mice administered with control antibody was increased, which shows that the refractory asthma is induced. In contrast, the number of neutrophils was significantly reduced in the group of mice administered with the anti-CXCL2 antibody, which suggests alleviation of refractory asthma. Furthermore, no significant decrease was found in the number of other cells such as macrophages and lymphocytes, which indicates a selective and significant decrease only in the number of neutrophils. Since CXCL2 is located at a downstream part of the signal transduction pathway, it can be expected that only neutrophils can be selectively reduced, which can result in less side effects.

<Histopathological Analysis of Lung Tissues>

Mouse lung tissues obtained from the lung samples were fixed with a 10% formalin solution (manufactured by Nacalai Tesque Inc.) and then embedded in paraffin. The sections were subjected to HE staining (hematoxylin and eosin staining). The results are shown in FIGS. 2(a) and 2(b). FIGS. 2(a) and 2(b) are images showing pathological lung tissue obtained in the group of mice administered with the control antibody and in the group of mice administered with the anti-CXCL2 antibody, respectively, which clearly indicates the inflammation of refractory asthma is properly induced in the lung tissues. In the figures, the scale bar indicates 10 μm. In FIG. 2(a), the arrows (Δ) indicate neutrophils.

The results in FIG. 2(a) show that, in the group of mice administered with the control antibody, neutrophils significantly infiltrated into the airway and erythrocytes were observed, which indicates bleeding and advanced inflammation. On the other hand, the results in FIG. 2(b) show that, in the group of mice administered with the anti-CXCL2 antibody, neither neutrophilic infiltration nor erythrocytes were observed, which indicates that no inflammation occurred. These results suggest that the administration of the anti-CXCL2 antibody dramatically inhibited the development of refractory asthma. The results shown in the sections <Counting of Different Types of Immune Cells in Bronchoalveolar Lavage Fluid>and <Histopathological Analysis of Lung Tissues>demonstrate that anti-CXCL2 antibodies can function as prophylactic or therapeutic agents for refractory asthma. As a surprising result, it was also found that, among different chemokines for neutrophils, selective inhibition of only CXCL2 could inhibit both neutrophilic infiltration and inflammation.

Furthermore, since it is apparent that anti-CXCL2 antibodies can function as prophylactic or treat agents for refractory asthma, it is suggested that antibodies (anti-CXCR2 antibodies) that selectively bind to CXCR2, a receptor for which CXCL2 is a ligand, can also function as prophylactic or therapeutic agents for refractory asthma.

### Sequence Listing

ATF-362PCT_ST25.txt

Having thus described the present teachings, it is now claimed:

1. A screening method comprising screening a prophylactic or therapeutic agent for refractory asthma using, as an indicator, at least one selected from the group consisting of inhibition of CXCL2 or CXCR2 protein activity, inhibition of CXCL2 or CXCR2 gene expression, and inhibition of CXCL2 or CXCR2 protein expression.

2. The method according to claim 1, wherein the indicator is inhibition of CXCL2 or CXCR2 protein activity, inhibition of CXCL2 or CXCR2 gene expression, or inhibition of CXCL2 or CXCR2 protein expression in a refractory asthma-induced animal administered with a test substance compared with a refractory asthma-induced animal not administered with the test substance.

3. The method according to claim 1, wherein the refractory asthma is neutrophil-dominant refractory asthma.

4. A prophylactic or therapeutic method for refractory asthma, comprising: administering an inhibitor of CXCL2 or CXCR2 protein activity, an inhibitor of CXCL2 or CXCR2 gene expression, or an inhibitor of CXCL2 or CXCR2 protein expression to a patient.

5. The method according to claim 4, wherein the inhibitor is an antibody or aptamer that selectively binds to a CXCL2 protein or a CXCR2 protein.

6. The method according to claim 4, wherein the refractory asthma is neutrophil-dominant refractory asthma.

7. The method according to claim 2, wherein the refractory asthma is neutrophil-dominant refractory asthma.

8. The method according to claim 5, wherein the refractory asthma is neutrophil-dominant refractory asthma
